(19) United States
(12) Patent Application Publication
(10) Pub. No.: US 2026/0027229 A1
(43) Pub. Date: Jan. 29, 2026

(54) ARTIFICIAL INTELLIGENCE-GUIDED PROTEIN DESIGN FOR NANOVESICLE ANALYSIS AND ENGINEERING

(71) Applicant: ACCURE HEALTH INC., Cambridge, MA (US)

(72) Inventors: Liyun SANG, Cambridge, MA (US); Qiang LIU, Cambridge, MA (US)

(73) Assignee: ACCURE HEALTH INC., Cambridge, MA (US)

(21) Appl. No.: 19/274,003

(22) Filed: Jul. 18, 2025

Related U.S. Application Data
(63) Continuation of application No. PCT/US2024/016921, filed on Feb. 22, 2024.
(60) Provisional application No. 63/447,384, filed on Feb. 22, 2023.
(60) Provisional application No. 63/517,149, filed on Aug. 2, 2023.

(51) Int. Cl. A61K 47/69; A61K 9/50; A61K 47/64; C07K 14/00

(52) U.S. Cl. CPC A61K 47/6901; A61K 9/5068; A61K 47/64; C07K 14/001; C07K 2319/02

(57) ABSTRACT

This disclosure provides an engineered delivery system, including a bacterial membrane vesicle derived from bacteria and one or more non-bacterial proteins for anchoring to the bacterial membrane vesicle.

### CROSS-REFERENCE TO RELATED APPLICATIONS

This application is a continuation of International Application No. PCT/CN2024/016921, filed on Feb. 22, 2024, which claims the benefit of priorities to U.S. Provisional Application No. 63/447,384, filed on Feb. 22, 2023, and U.S. Provisional Application No. 63/517,149, filed on Aug. 2, 2023. The entire contents of each of the above-referenced applications are expressly incorporated herein by reference.

### REFERENCE TO A SEQUENCE LISTING

A Sequence Listing conforming to the rules of WIPO Standard ST.26 is hereby incorporated by reference. The electronic document, created on Feb. 22, 2024, is titled “Accure-2024-02-22-SEQL.xml”, and is 73,446 bytes in size.

### BACKGROUND

The success of mRNA Covid vaccines has renewed interest in mRNA as a means of delivering therapeutic proteins. In many cases, it is necessary to deliver mRNA to specific targets, cells, or tissues, and maintain a durable and high level of expression in target cells. It is critical to develop safe and more effective delivery technologies to unlock the promise of targeted mRNA therapeutics, for the treatment of both rare and common diseases.

### SUMMARY

Embodiments of the disclosure provide an engineered delivery system. The engineered delivery system includes a bacterial membrane vesicle derived from bacteria and one or more non-bacterial proteins for anchoring to the bacterial membrane vesicle.

In some embodiments, the one or more non-bacterial proteins include a mammalian membrane-associated protein or a fragment thereof.

In some embodiments, the one or more non-bacterial proteins are further linked to a polypeptide binder, either directly or by a linker.

In some embodiments, the polypeptide binder is displayed on an outer side of the vesicle membrane.

In some embodiments, the linker is a glycine-serine linker.

In some embodiments, the glycine-serine linker is GGGGS.

In some embodiments, the polypeptide binder is a synthetic polypeptide.

In some embodiments, the polypeptide binder includes a nucleic acid binding domain.

In some embodiments, the polypeptide binder includes an amino acid sequence as set forth in SEQ ID NO: 4, 5, 22, 23, 24, or 61.

In some embodiments, the bacteria are gram-negative bacteria.

In some embodiments, the gram-negative bacteria are Escherichia coli.

In some embodiments, the one or more non-bacterial proteins include mammalian protein voltage-dependent anion-selective channel 1 (VDAC1), mitochondrial carrier homolog 2 (MTCH2), or acyl-CoA synthetase long-chain family member 1 (ACSL1), or a fragment thereof.

In some embodiments, the mammalian protein comprises an amino acid sequence as set forth in SEQ ID NO: 1, 2, or 3.

In some embodiments, the non-bacterial protein's full amino acid sequence is set forth in SEQ ID NO: 6, 7, 8, 9, 10, 11, 12, 13, 25, 27, 29, 31, 33, 35, 37, 39, 41, 43, 45, 47, 49, 51, 53, 55, 57, or 59.

In some embodiments, the engineered delivery system further includes a nucleic acid, a protein, a complex thereof, or a combination thereof within the bacterial membrane vesicle.

In some embodiments, the nucleic acid is mRNA, circular RNA, or antisense oligonucleotide.

Embodiments of the disclosure also provide a synthetic polypeptide binding to programmed death-ligand 1 (PD-L1). The synthetic polypeptide includes amino acid sequence as set forth in SEQ ID NO: 4, 5, 23, or 24, including conservative mutants thereof.

In some embodiments, the synthetic polypeptide is linked to a small molecule label, and the small molecule label is positioned either N-terminal or C-terminal to SEQ ID NO: 4, 5, 23, or 24.

In some embodiments, the synthetic polypeptide further includes a second polypeptide either N-terminal or C-terminal to SEQ ID NO: 4, 5, 23, or 24.

In some embodiments, the synthetic polypeptide further includes a bacterial signal peptide.

In some embodiments, the full amino acid sequence is set forth in SEQ ID NO: 14, 15, 16, 17, 18, 19, 20, 21, 28, 30, 34, 36, 40, 42, 46, 48, 52, 54, 56, 58, 60, 62, 63, or 64.

Embodiments of the disclosure further provide a synthetic polypeptide specifically binding to Claudin 18.2. The synthetic polypeptide includes amino acid sequence as set forth in SEQ ID NO: 22, including conservative mutants thereof.

In some embodiments, the synthetic polypeptide is further linked to a small molecule label either N-terminal or C-terminal to SEQ ID NO: 22.

In some embodiments, the synthetic polypeptide further includes a second polypeptide either N-terminal or C-terminal to SEQ ID NO: 22.

In some embodiments, the synthetic polypeptide further includes a bacterial signal peptide.

In some embodiments, the full amino acid sequence is set forth in SEQ ID NO: 26, 32, 38, 44, or 50.

Embodiments of the disclosure further provide a method for engineering microbial vesicles. The method includes computationally designing a membrane vesicles (EV) scaffold protein comprising a human membrane anchor peptide, a synthetic polypeptide binder, and a bacterial signal peptide domain; cloning the EV scaffold protein in a bacterial expression vector; expressing the EV scaffold protein in E. coli or other gram-negative bacteria; purifying the EV scaffold protein from bacteria expression culture; and validating the EV scaffold protein for target binding and cell uptake.

### BRIEF DESCRIPTION OF DRAWINGS

FIG. 1 shows the size and quantitation of Spirulina membrane vesicles (EV) with Nanoparticle Tracking Analysis (NTA). The mean size of Spirulina EV was about 177 nm.

FIG. 2 is a schematic illustrating the in vitro target binding TiMES assay. The assay is to validate if the engineered bacterial membrane vesicles (EV) bind to their target protein (e.g., PD-L1, Claudin 18.2) in vitro. The engineered bacteria EV expressing the designed scaffold binds to the target protein (e.g., PD-L1, Claudin 18.2) coated on the magnetic particles (MP), and subsequently labeled with a housekeeping EV marker for electrochemical detection using the TIMES assay and device, for example, as disclosed in U.S. Pat. No. 11,125,745 and U.S. Patent Application Publication No. 2021/0208169, both of which are hereby incorporated by reference in their entireties.

FIG. 3 is a schematic showing targeted delivery of RNA to mammalian cells using engineered microbial vesicles. The cellular uptake of the vesicle is facilitated by the interaction between the engineered vesicles and the target biomarker protein on the cell surface.

FIG. 4 is a schematic showing targeted delivery of RNA to mammalian gastric cells using engineered microbial vesicles expressing human gastric tissue-specific target biomarker protein (ATP4A). The uptake of the vesicle by the gastric cell is facilitated by the interaction between the engineered vesicles and ATP4B expressed on the cell surface.

FIG. 5 is a flowchart of an experiment workflow illustrating the design, production, and validation of engineered microbial membrane vesicles.

FIG. 6 is a schematic illustrating the design, production, isolation, RNA loading, and cellular uptake of the engineered microbial membrane vesicles, and the RNA expression in target cells.

FIG. 7 is a schematic illustrating the TIMES assays for the capture or detection of EV biomarkers using AI-designed protein binders.

FIG. 8 is a schematic and fluorescence microscopy image showing the detection of cellular PD-L1 expression using an AI-designed PD-L1 binder.

FIG. 9 is schematic and fluorescence microscopy images showing that an AI-designed PD-L1 binder can block the interaction between cellular PD-L1 and its antibody.

FIG. 10 is a graph showing the normalized PD-L1 binding signals from various engineered E. coli EVs and control samples.

FIG. 11 is a graph showing the normalized Claudin 18.2 binding signals from various engineered E. coli EVs and a control sample.

FIG. 12 illustrates schematic and fluorescence microscopy images showing the uptake of the engineered microbial membrane vesicles and the expression of eGFP mRNA in PD-L1 expressing target cells.

FIG. 13 is a flowchart showing the design and production of protein binders and their applications in analyte capture, detection, and blocking.

### DETAILED DESCRIPTION

Unless defined otherwise, all technical and scientific terms used herein have the same meaning as commonly understood by one of ordinary skill in the art to which the disclosure pertains. The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only, and is not intended to be limiting.

As used in this specification and the appended claims, the singular forms “a,” “an,” and “the” include plural referents unless the context clearly dictates otherwise. Thus, for example, reference to “an analyte” includes a plurality of such analytes, and the like.

The term “about” as used herein indicates the value of a given quantity varies by ±10% of the value.

Unless clearly indicated otherwise, the ranges listed herein are inclusive.

A variety of additional terms are defined or otherwise characterized herein.

The success of mRNA Covid vaccines has renewed interest in mRNA as a means of delivering therapeutic proteins. However, the development of mRNA therapeutics presents additional challenges. Compared to mRNA vaccines, mRNA therapeutics require as much as a 1,000-fold higher level of protein to reach a therapeutic threshold. In many cases, it is necessary to deliver mRNA to specific targets, cells, or tissues, and maintain a durable and high level of expression in target cells. Most of the mRNA therapeutics under development rely on lipid nanoparticles (LNPs). However, LNPs delivered via systemic administration largely accumulate in the liver; efficient delivery to other solid organs remains challenging. Another major hurdle associated with LNPs in circulation is immunogenicity. Even with advanced LNPs, repeated dosing for chronic disease activates innate immunity and leads to cytotoxicity and rapid clearance of LNPs. On the other hand, oral delivery is the most widely used form of drug administration, but the gastrointestinal (GI) tract presents numerous barriers to LNP-based RNA delivery. Notably, acidic pH and the formation of coronas in GI fluids significantly impact the stability and cellular uptake of LNPs. Therefore, it is critical to develop safe and more effective delivery technologies to unlock the promise of targeted mRNA therapeutics, for the treatment of both rare and common diseases.

Extracellular vesicles (EVs) are naturally derived, lipid-bound nanoparticles involved in cell-to-cell communication across all kingdoms of life. They carry a variety of biological cargoes (e.g., DNA, RNA, protein) and transfer them between cells through endocytosis and exocytosis. EVs have thus been explored as drug carriers to deliver therapeutic payloads to specific cells or tissues, harnessing their intrinsic tissue-homing capabilities. Compared with conventional synthetic carriers (e.g., LNPs), EVs possess multiple advantages, such as biocompatibility, relatively higher stability in biological fluids, and low immunogenicity. Built on promising safety profiles, a number of EV-based therapeutic interventions have entered Phase 1 or Phase 2 clinical trials for various conditions, including tissue regeneration, stroke, and cancer (clinicaltrials.gov/). Despite extensive research, the broader clinical translation of EVs as drug carriers is still hampered by a lack of robust EV engineering strategies, low-cost production systems, and effective analytical methods.

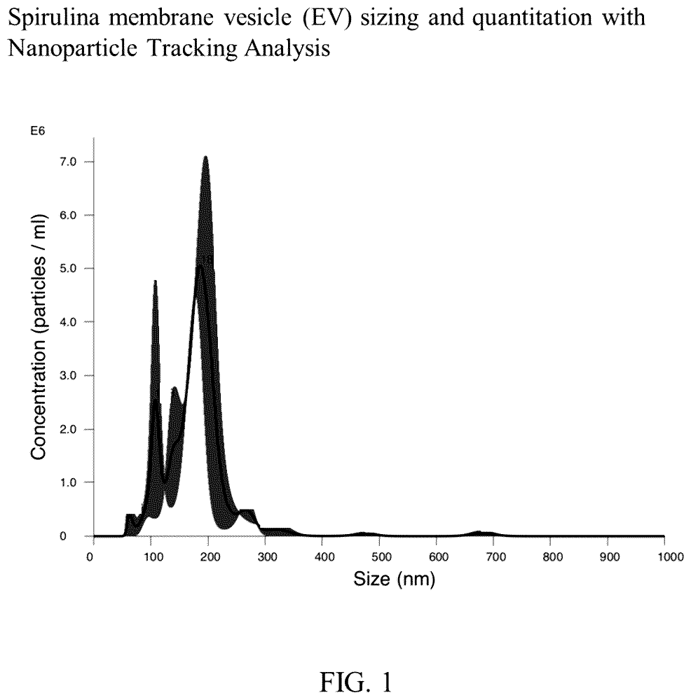

Microbial systems, including non-pathogenic bacteria, probiotics, and micro algae are cost-effective systems for manufacturing EV. As an example, Escherichia coli (E. coli) is a gram-negative bacterium commonly found in the lower intestine of mammalian organisms. Many E. coli strains are part of the normal microbiota of the gut and are harmless or even beneficial to humans. As another example, the Arthrospira platensis, commonly known as spirulina, is an edible, gram-negative cyanobacterium consumed worldwide for its high protein content and other nutritional benefits. Bioencapsulation within spirulina biomass protects the therapeutic cargo from the low pH and high pepsin gastric environment. Oral delivery of a spirulina-expressed therapeutic antibody demonstrated safety for human administration in a phase 1 clinical trial. Non-pathogenic E. coli and Spirulina are potentially advantageous systems to manufacture EVs as drug carriers: i) they combine the safety of a non-pathogenic host with the high productivity and low cost of microbial platforms; ii) E. coli and spirulina cells release EVs in abundance with a size distribution similar to human EVs (FIG. 1). In addition, the spirulina outer membrane is chemically distinct from other gram-negative bacteria and barely contains any pro-inflammatory endotoxin. Spirulina-derived EVs are not only safe vehicles for oral delivery but may be more easily purified for systemic delivery due to low immunogenicity.

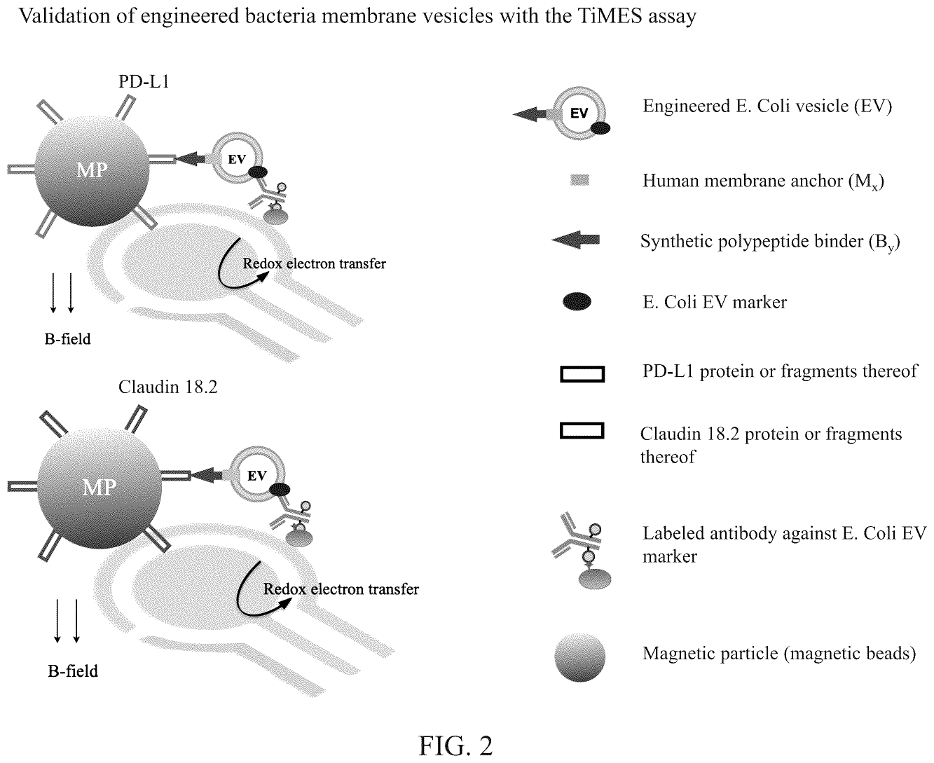

Most of the human EV membrane anchoring proteins cannot be readily expressed in microbial hosts due to different membrane targeting pathways in microbial systems and a lack of specific post-translational modification mechanisms. Addressing the unmet need for EV engineering and analysis, this disclosure discloses two recently developed technologies to advance EV-based drug delivery. i) Sequence-and structure-based AI algorithms for designing target-specific EV protein scaffolds. Considerable progress has been made in folding and designing proteins using deep learning methods. Diffusion models showed unprecedented success in image and language generative modeling, and also achieved outstanding performance on topology-constrained protein design, protein binder design, and motif scaffolding. The design speed and success rates have improved significantly over the past years, and de novo designed proteins can now achieve high binding affinity and specificity to both structured and flexible targets. An EV biomarker database has been established, including EV proteins that are largely stable in human bio-fluids, as well as tissue-enriched or disease-specific EV proteins. These resources allow refining AI parameters for designing compact and target-specific EV protein scaffolds. ii) An advanced EV analytical system “TIMES” has been developed, based on integrated Magneto-Electronic Sensing technology, for example, as disclosed in U.S. Pat. No. 11,125,745 and U.S. Patent Application Publication No. 2021/0208169, both of which are hereby incorporated by reference in their entireties. The TIMES system carries out automated EV isolation and detection in a single platform, and offers distinct advantages: (a) EVs can be analyzed directly from complex media without filtration or centrifugation; (b) it has a superior analytical performance with a limit-of-detection (LOD) of ˜10⁴ EVs (˜1,000-fold more sensitive compared to ELISA) and a dynamic range spanning 4 orders of magnitude; (c) multiple EV biomarkers can be analyzed in parallel and the total assay time is <1 hour. In this disclosure, the advantages of AI-powered EV design and TIMES EV analysis with spirulina-based production are combined to accelerate the development of target-or tissue-specific drug delivery. FIG. 2 shows a schematic illustrating the in vitro target binding TiMES assay. The assay is to validate if the engineered bacterial membrane vesicles (EV) bind to their target protein (e.g., programmed death-ligand 1 (PD-L1), Claudin 18.2) in vitro. The engineered bacteria EV expressing the designed scaffold binds to the PD-L1 protein coated on the magnetic particles (MP), and subsequently labeled with a housekeeping EV marker for electrochemical detection using the TIMES assay and device.

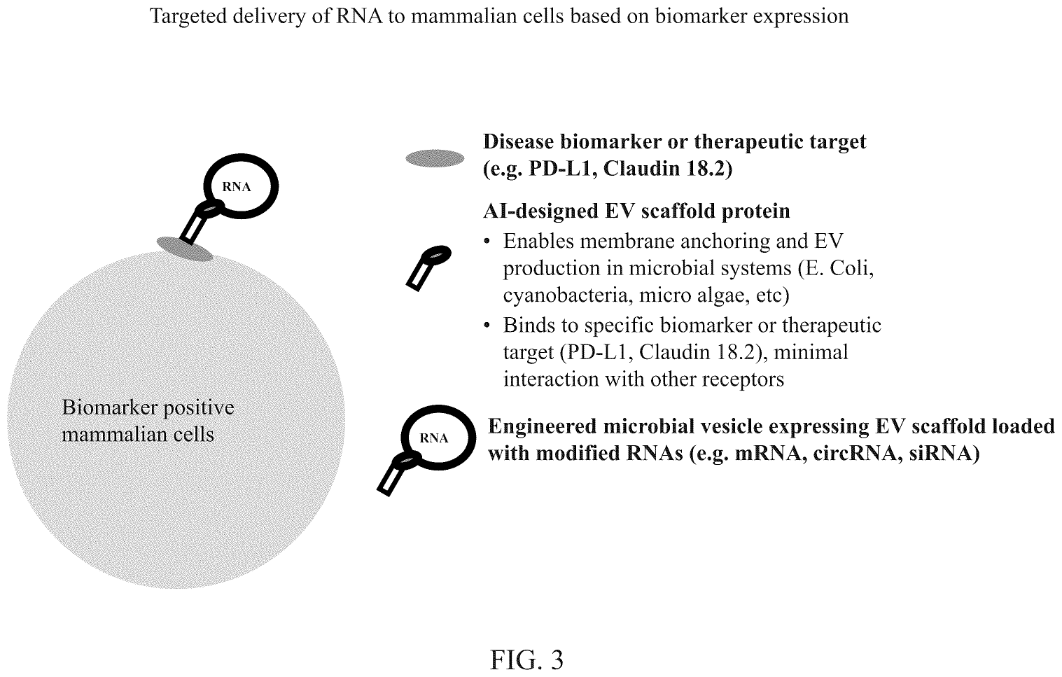

As an example, EV scaffolds have been engineered to facilitate targeted RNA delivery to PD-L1 positive cells (FIG. 3), with both technical and commercial considerations: (i) PD-L1 is a well-established therapeutic target for various solid tumors, including GI cancers that can be treated via both systemic and oral routes; (ii) a wealth of cell lines and other reagents are available to help assess and benchmark effectiveness and specificity for PD-L1 targeting; (iii) there are clinical and commercial interests to develop combination therapies with programmed cell death protein 1 (PD-1) inhibitor (anti-PD1)/PD-L1 drugs. The PD-L1 targeting EV may provide a strategy to combine anti-PD1/PD-L1 antibody with a new mRNA tumor suppressor drug.

As another example, EV scaffolds have been engineered to facilitate targeted RNA delivery to Claudin 18.2 positive cells (FIG. 3): (i) Claudin 18.2 is an emerging therapeutic target for various solid tumors, including gastric cancer and pancreatic cancers that can be treated via systemic or oral routes; (ii) there are clinical and commercial interests to develop combination therapies with anti-Claudin 18.2 drugs. The Claudin 18.2 targeting EV may provide a strategy to combine anti-Claudin 18.2 antibody with a new mRNA tumor suppressor drug.

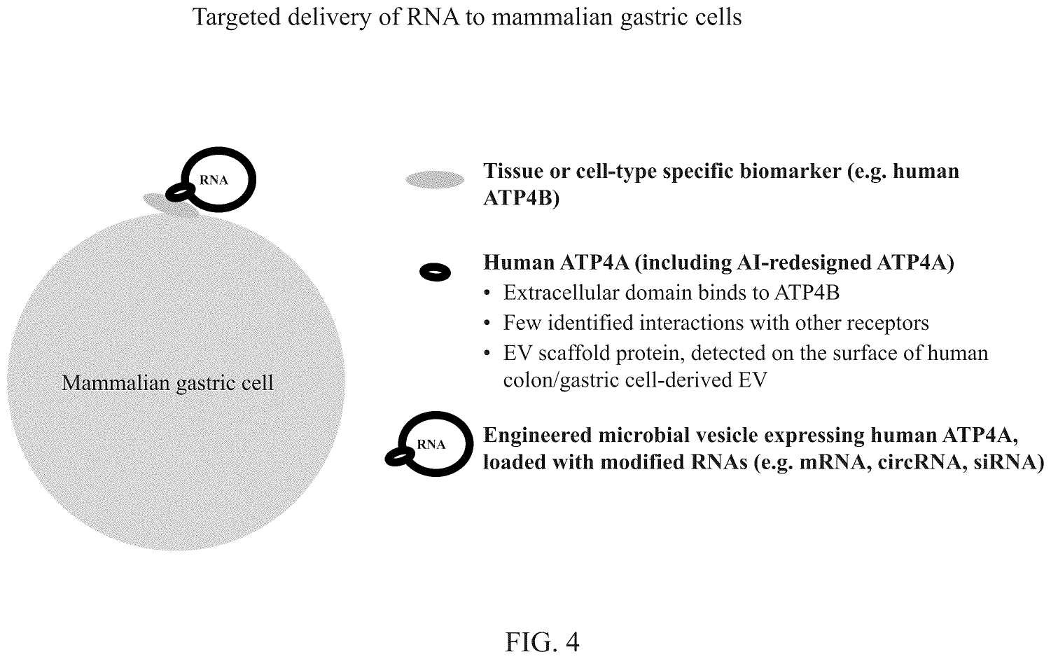

In yet another example, EV scaffolds can be engineered to facilitate targeted RNA delivery to ATP4B positive cells (FIG. 4). ATP4B is a gastric tissue-specific protein biomarker. Engineered microbial EV expressing the extracellular domain of human ATB4A or expressing an AI-redesigned version of ATB4A (e.g., hallucinated ATB4A) can be utilized to deliver therapeutic nucleic acids (e.g., gene editors, RNAs for tumor suppressors, proteins degraders) to normal gastric tissue or gastric cancer cells (FIG. 4).

The PD-L1, Claudin 18.2, or ATP4B targeting EV is merely an example. The microbial EV platform can be readily used for targeting additional cell types (e.g., EGFR-mutant tumor cells, T cells, astrocytes) and delivering a variety of RNA payloads (e.g., RNAs for tumor suppressors, proteins degraders, chimeric antigen receptors, gene editors).

In this disclosure, a method has been developed by combining AI-powered vesicle design and analysis with a low-cost microbial production and delivery system to enable target-or tissue-specific drug delivery. Key features and competitive advantages are discussed below and in Table 1.

TABLE 1
Competitive advantages of engineered microbial EV for RNA delivery

| | | Payload | | Manufacturing | |
|---|---|---|---|---|---|
| Carrier type | Targeted uptake | loading | Toxicity/immunogenicity | scalability | Cost |
| Lipid nanoparticle | Low-medium | Passive | Medium-high | High | $$ |
| (LNP) | | | | | |
| Native human EV | Tropism, not | Passive | Low | Low | $$$ |
| | programmable | | | | |
| Engineered human | Programmable | Passive | Low | Low | $$$$ |
| EV | | and | | | |
| | | active | | | |
| Accure engineered | Programmable | Passive | Medium | High | $$ |
| probiotic EV | | and | | | |
| | | active | | | |
| Accure engineered | High | Passive | Low | High | $$ |
| microalgae EV | (Programmable) | and | | | |
| | | active | | | |

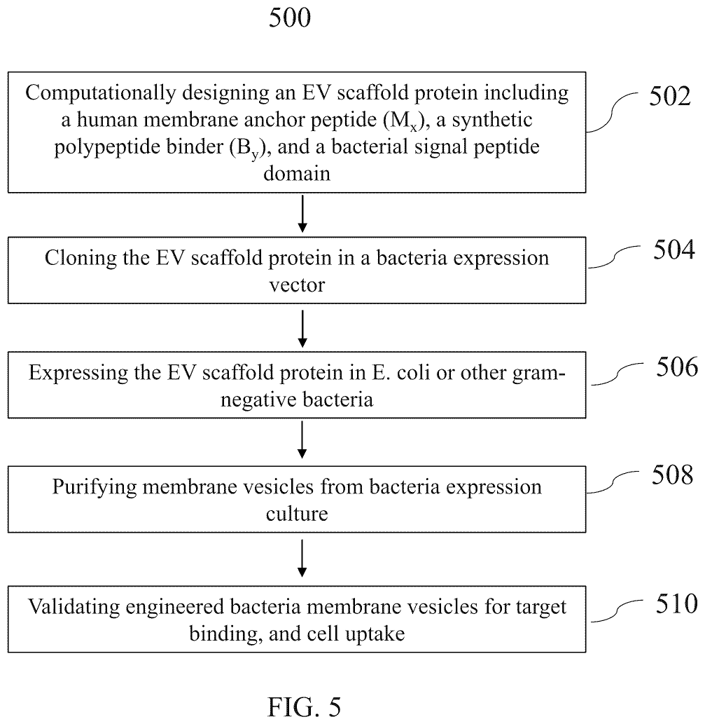

FIG. 5 is a flowchart of a method illustrating the design, production, and validation of engineered microbial membrane vesicles. It is understood that the operations shown in the method may not be exhaustive and that other operations can be performed as well before, after, or between any of the illustrated operations. Further, some of the operations may be performed simultaneously, or in a different order than shown in FIG. 5.

Referring to FIG. 5, method 500 starts at operation 502, which involves computationally designing an EV scaffold protein including a human membrane anchor peptide, a synthetic polypeptide binder, and a bacterial signal peptide domain. In some embodiments, deep learning-based structure prediction, diffusion generative models, and EV biomarker database may be used to design new EV scaffold proteins with high accuracy and speed. This approach allows for: a) rapid design of candidate EV scaffolds to promote membrane anchoring and vesicle release from microbial hosts, b) effective loading of RNA and protein payloads, and c) in silico screening of candidate designs to improve targeted uptake and minimize off-target interactions.

At operation 504, the EV scaffold protein is cloned in an expression vector. In some embodiments, native and engineered EVs of human or bacterial origin may be used as drug carriers for RNA and protein therapeutics. Preliminary studies suggest that human-cell (e.g., blood cell, HEK293T)-derived EVs are largely safe without notable adverse effects. However, manufacturing challenges (including drug loading efficiency, batch-to-batch variations, and cost), have limited their scalability. Engineering and production of microbial EVs may be more readily scalable. However, most microbial engineering strategies utilize viral proteins for membrane targeting and anchoring, which may result in toxicity and immunogenicity. The method disclosure herein relies on unique mammalian or humanized recombinant proteins to minimize toxicity and immunogenicity.

At operation 506, the EV scaffold protein is expressed in E. coli or other gram-negative bacteria.

At operation 508, the EVs that express the engineered scaffold protein are purified from bacteria expression culture.

At operation 510, the EVs are validated for target binding and cell uptake. In some embodiments, the automated analytical system (TIMES) may be used to rapidly monitor the quantity and stability of engineered microbial EVs during production and post-administration. When used in EV quality assessment, both cell targeting and RNA loading properties of the engineered EVs can be monitored in nearly real-time (˜1 hour from sample to answer). In addition, following the intracellular release of therapeutic payloads (e.g., in tumor, immune, or gut cells), treatment responses can also be analyzed non-invasively using human cell-derived EVs.

As an example, state-of-the-art protein design algorithms and parameters (e.g., RFdiffusion, ProteinMPNN) may be used to enable modular designs of EV scaffold proteins. Membrane anchoring, payload (RNA) binding, and target binding structures may be first designed independently to improve computational throughput and then evaluated in combination for structure stability and target binding. Designs may be refined by successive noising and denoising (partial diffusion). A design may be classified as successful if the Alphafold2 (AF2) Predicted Aligned Error (pAE) between the designed protein and target (e.g., PD-L1)<10; the Root Mean Square Deviation (RMSD) between the designed protein and the AF2 prediction<2 Å, and AF2 predicted local distance difference test (pLDDT)>80. As an example, EV protein scaffolds targeting PD-L1, or Claudin 18.2 have been designed.

In some embodiments, computational protein-protein interaction analysis may be performed to assess target binding as well as non-specific interactions. The successful designs from the step above may be evaluated for target binding using protein complex prediction algorithms. The top 50 best-performed designs (based on pDockQ, IPTM, pAE scores may be further evaluated for non-specific interactions with a proprietary database of additional targets, including EV and receptor proteins highly expressed in normal tissues or immune cells, as well as proteins associated with drug toxicities.

In some embodiments, computational tools (e.g., patchwork) may be used to evaluate the designed proteins and assess how pH may affect their stability and binding interface.

Codon-optimized genes encoding the designed protein may be synthesized and cloned into the E. coli protein expression vector. Following plasmid transformation and protein induction in E. coli cells (e.g., Lemo21), EVs may be harvested and purified from the bacterial supernatants using the ExoBacteria OMV Isolation Kit or using ultracentrifugation. Lemo 21 is merely an example. In practice, other non-pathogenic bacteria stains may also be used, including but not limited to BL21 (DE3), E. coli Nissle 1917, Lactobacillus, Bifidobacterium, and Bacillus licheniformis.

In some embodiments, codon-optimized genes encoding the designed proteins may be cloned into integrating vectors for spirulina transformation. When engineered spirulina strains are near complete segregation, the whole cell biomass and cell culture supernatant may be harvested using the ExoBacteria OMV Isolation Kit or using ultracentrifugation. Spirulina is merely an example. In practice, other micro algae strains may also be used, including but not limited to Syncchocystis sp. PCC 6803, Prochlorococcus marinus subsp. pastoris str. CCMP1986, Cyanophora paradoxa, and Tetraselmis chuii.

In some embodiments, codon-optimized genes encoding one or more non-bacteria proteins may be synthesized and cloned into the E. coli protein expression vector. Following plasmid transformation and protein induction in E. coli cells (e.g., Lemo21), EVs may be harvested and purified from the bacterial supernatants using the ExoBacteria OMV Isolation Kit or using ultracentrifugation. Lemo 21 is merely an example. In practice, other non-pathogenic bacteria stains may also be used, including but not limited to BL21 (DE3), E. coli Nissle 1917, Lactobacillus, Bifidobacterium, and Bacillus licheniformis. In some embodiments, the engineered delivery system will comprise a bacterial membrane vesicle (EV) derived from bacteria; and one or more non-bacterial proteins for anchoring to the bacterial membrane vesicle. In some embodiments, the one or more non-bacteria proteins comprise a mammalian membrane-associated protein or a fragment thereof.

In some embodiments, the mammalian membrane-associated protein is further linked to a polypeptide binder, either directly or indirectly by using a linker. The polypeptide binder may be a synthetic polypeptide, or the polypeptide binder may include a nucleic acid binding domain. In some embodiments, the polypeptide binder includes an amino acid sequence as set forth in SEQ ID NO: 4, 5, 22, 23, 24, or 61.

In some embodiments, the polypeptide binder may be displayed on an outer side of the vesicle membrane. The membrane orientation of the binder is determined by the protein sequence and structure of the membrane-associated protein and the binder. The linker may be a glycine-serine linker, and the glycine-serine linker may be GGGGS. Codon-optimized genes encoding the fused protein may be synthesized and cloned into an E. coli protein expression vector, to produce the fused protein and the membrane vesicles displaying the fused protein.

In some embodiments, gram-negative bacteria may be used for engineering EV as a delivery system. Particularly, the gram-negative bacteria may be Escherichia coli.

In some embodiments, the one or more non-bacteria proteins may include mammalian proteins such as voltage-dependent anion-selective channel 1 (VDAC1), mitochondrial carrier homolog 2 (MTCH2), or acyl-CoA synthetase long-chain family member 1 (ACSL1), or a fragment thereof. The mammalian protein may include an amino acid sequence as set forth in SEQ ID NO: 1, 2, or 3.

In some embodiments, the non-bacterial protein's full amino acid sequence is set forth in SEQ ID NO: 6, 7, 8, 9, 10, 11, 12, 13, 25, 27, 29, 31, 33, 35, 37, 39, 41, 43, 45, 47, 49, 51, 53, 55, 57, or 59.

In some embodiments, the engineered EV may further include a nucleic acid (such as DNA or RNA), a protein (such as an enzyme or antibody), their complexes, or any combination of these components. The nucleic acid may be mRNA, circular RNA, or antisense oligonucleotide.

Purified EVs may be analyzed using an automated TiMES device. The assay may use magnetic particles (MPs) coated with the target protein (e.g. PD-L1, or Claudin 18.2) to capture bacteria EV that express the designed targeting scaffold. The antibody against a housekeeping bacteria EV marker (e.g., Enolase, OmpA, GroEL) may be introduced and conjugated with an oxidizing enzyme (horseradish peroxidase, HRP). The MP-EV complexes may be mixed with chromogenic electron mediators (3,3′,5,5′-tetramethylbenzidine, TMB), and magnetically concentrated on top of an electrode; HRP catalyzes the oxidation of TMB, and the oxidized TMB is then reduced by receiving electrons from the electrode, which generates electrical current as an analytical readout (FIG. 2). The net signal difference (A) between the engineered EV sample and the control sample (EVs purified from control E. coli) may be obtained. The A/may be further normalized to the total bacteria EV concentration in the cell culture.

In some embodiments, binding of engineered microbial vesicles to the target protein may also be validated with conventional immunoassays such as enzyme-linked immunosorbent assay (ELISA), or with Flow cytometry using a similar approach as described above.

In some embodiments, to assess the stability of engineered microbial EV in biological fluids, purified EVs may be challenged by immersing them in human plasma or gastric fluid, and re-measure the EV concentration and test if their binding to the target protein has been impacted by these biological fluids.

In some embodiments, RNAs (e.g., cGFP mRNA, cGFP circular RNA) may be loaded to the engineered microbial EVs via Electroporation. Modified eGFP mRNA or circRNA may be synthesized by in vitro transcription and encapsulated into microbial EV using an optimized electroporation protocol, to preserve EV membrane integrity and mRNA stability. Following electroporation, RNase may be applied to remove exogenous mRNA. In some embodiments, RNAs may be loaded to the surface of microbial EVs via RNA-protein binding, namely “RBD backpack”. The RNAs that carry recognition sequences for specific RNA binding domains (RBD) may be synthesized by in vitro transcription, and loaded to microbial EV through binding to the respective RBD on the designed EV scaffold protein. In some embodiments, modified eGFP mRNA or circRNA may be synthesized by in vitro transcription and encapsulated into microbial EV using sonoporation. eGFP RNAs are the only examples. In practice, a variety of RNA payloads may be loaded to the engineered microbial EVs, including but not limited to RNAs for tumor suppressors, protein degraders, chimeric antigen receptors, and gene editors.

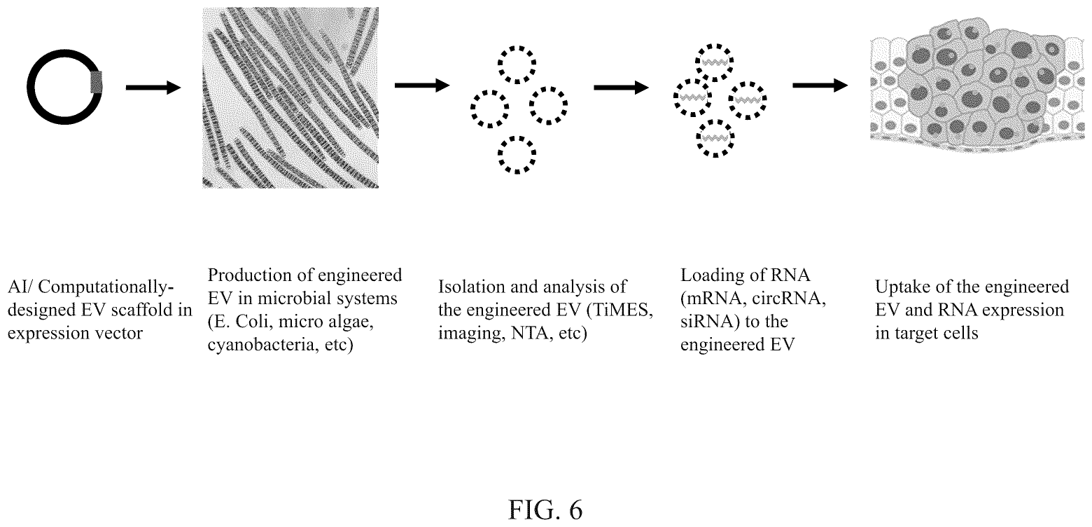

In some embodiments, engineered, RNA-loaded microbial EV may be administered to human cell lines expressing high or low levels of the target protein (proteinatlas.org). As an example shown in Table 2, the eGFP mRNA or circular RNA may be loaded to microbial EV as described above or added directly to the cell culture media without any carriers. Expression of eGFP may be measured by imaging and/or flow cytometry every 24 hrs until day 7 after RNA administration, to assess the level and duration of RNA expression in target cells. The delivery efficiency e is defined as: e=Percentage of eGFP-positive cells (P)×Mean fluorescence intensity (MFI). To further optimize cellular uptake efficiency, different RNA-EV ratios and EV-cell ratios may be tested, to estimate the lowest EV and RNA doses that can achieve durable RNA expression in target cells (FIG. 6).

TABLE 2
Microbial EV cellular uptake experiment design

| | Cell surface | RNA | RNA loading |
|---|---|---|---|
| Target cells | PD-L1 level | payload | method |
| Colon cancer | High (+++) | eGFP mRNA | Engineered EV (Electroporation) |
| (RKO) | | eGFP circRNA | Engineered EV (RBD backpack) |
| | | eGFP mRNA | LNP |
| Colon cancer | Not | eGFP mRNA | Engineered EV (Electroporation) |
| (NCI-H508) | detectable (−) | eGFP circRNA | Engineered EV (RBD backpack) |
| | | eGFP mRNA | LNP |
| Non- | Not | eGFP mRNA | Engineered EV (Electroporation) |
| cancerous | detectable (−) | eGFP circRNA | Engineered EV (RBD backpack) |
| (HEK293) | | eGFP mRNA | LNP |

In some embodiments, the functionality and safety of engineered microbial EVs may be tested in wide type and cell line-derived xenograft (CDX) mouse models. The cell lines to generate the CDX mouse model include but not limited to RKO colon cancer cells, HCC4006 lung cancer cells, Hs 746T gastric cancer cells, SNU-423 liver cancer cells, Panc 08.13 pancreatic cancer cells, HuP-T4 pancreatic cancer cells. Native or engineered microbial EVs carrying GFP mRNAs or circRNA may be mixed with excipient (e.g., spirulina powder) and given to mice via oral administration. The expression of GFP in cancer cells and gut cells (target or off-target) may be measured via intravital fluorescence microscopy. Alternatively, native or engineered bacteria EVs carrying GFP RNAs may be purified and given to mice via systemic or intranasal administration. The expression of GFP in target (or off-target) cells may be measured via intravital fluorescence microscopy. The serum cytokines and the histopathology of liver and kidney may be evaluated to determine toxicity.

In some embodiments, native or engineered bacterial EVs carrying therapeutic RNAs (e.g., RNAs for tumor suppressors, protein degraders, chimeric antigen receptors, gene editors) may be given to mice (or clinical study participants) via oral, systemic, or intranasal administration. The toxicity and therapeutic effects may be evaluated.

In some implementations, the computationally designed protein scaffold or binders may be used in in vitro analytical assays. In some embodiments, the protein scaffold or binder further includes a second polypeptide which is positioned either N-terminal or C-terminal to SEQ ID NO: 4, 5, 22, 23, or 24. The protein scaffold or binder may further include a bacterial signal peptide. The full amino acid sequence of the PD-L1 binding polypeptide may be set forth in SEQ ID NO: 14, 15, 16, 17, 18, 19, 20, 21, 28, 30, 34, 36, 40, 42, 46, 48, 52, 54, 56, 58, 60, 62, 63, or 64. The full amino acid sequence of the Claudin 18.2 binding polypeptide may be set forth in SEQ ID NO: 26, 32, 38, 44, or 50. Codon-optimized genes encoding the polypeptide sequences may be synthesized and cloned into the pET-29b(+) E. coli plasmid expression vectors. Plasmids may be transformed into chemically competent E. coli Lemo21 cells [or BL21 Star™ (DE3) cells]. Bacteria may be cultured at 37° C. (or 30° C.) in cultures of lysogeny broth (LB) supplemented with 50 μg/mL of kanamycin and 30μg/ml chloramphenicol. Cells may then be grown at 37° C. (or 30° C., 15° C.) in cultures of Terrific Broth (TB) supplemented with 50 μg/mL of kanamycin, 30 μg/ml chloramphenicol, 2 mM MgSO₄, and 0.5 mM L-rhamnose, until OD600 reaches 0.4-0.6, before induction with IPTG for 15˜20 hrs. Cells may be harvested by centrifugation at 4300 g at 4° C. and lysed, and then treated with DNasel and protease inhibitors. Clarified lysate supernatants may be batch bound with equilibrated Ni-NTA resin and subsequently washed thrice with a wash buffer. The proteins may be further purified by size exclusion chromatography, and characterized by SDS-PAGE.

In some implementations, the computationally-designed protein binder may be used as a capture or detection agent in immunoassays. Purified binder protein (e.g., PD-L1 binder) may be immobilized to plastic surfaces, magnetic particles, or biosensors to capture free target proteins (e.g., PD-L1), cells, or extracellular vesicles that express the target protein (e.g., PD-L1). These captured cells, vesicles, or proteins may subsequently be labeled with another protein binder or antibody for signal detection. In some embodiments, target-expressing cells, vesicles, or free proteins may first be captured using a capture antibody and then labeled with the designed protein binder (including biotinylated, or tag-linked binder) for signal detection. In some embodiments, the PD-L1 or Claudin 18.2 binding polypeptide is linked to a small molecule label. Particularly, the PD-L1 binding polypeptide is linked to the small molecule label which is positioned either N-terminal or C-terminal to SEQ ID NO: 4, 5, 22, 23, or 24. In some embodiments, the small molecule label can be a biotin molecule, which is linked to the polypeptide through chemical or enzymatic biotinylation. In some embodiments, the PD-L1 or Claudin 18.2 binding polypeptide, further fused to a second polypeptide (e.g., his-tag, MBP-tag, Avi-tag, cGFP-tag) either N-terminal or C-terminal to SEQ ID NO: 4, 5, 22, 23, or 24. These computationally-designed protein binders are particularly valuable in immunoassays, when i) no functional antibody is available, or ii) only one functional antibody is available. The computationally-designed protein binders may be advantageous when non-specific antibody interaction is a concern.

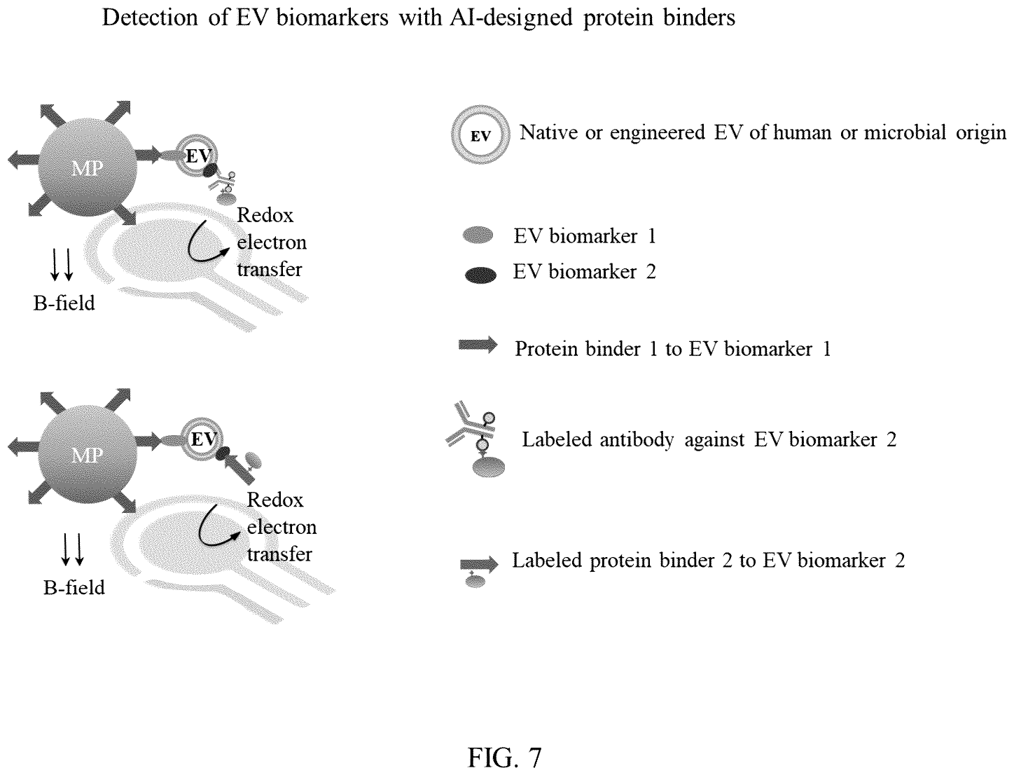

In some implementations, the computationally-designed protein binder may be used as a capture or detection agent in magneto-electrochemical sensing assay. Purified binder protein (e.g., PD-L1 binder) may be immobilized to magnetic particles (MP) to capture free target proteins (e.g., PD-L1), or extracellular vesicles that express the target protein (e.g., PD-L1). These captured vesicles or proteins may subsequently be labeled with another protein binder or antibody for signal detection (FIG. 7). In some embodiments, vesicles or free proteins may first be captured using a capture antibody and then labeled with the designed protein binder (including biotinylated, or tag-linked binder). MP-EV complexes may then be magnetically concentrated on top of a sensing electrode; redox reactions and electron transfer from the electrode generated electrical current as an analytical readout.

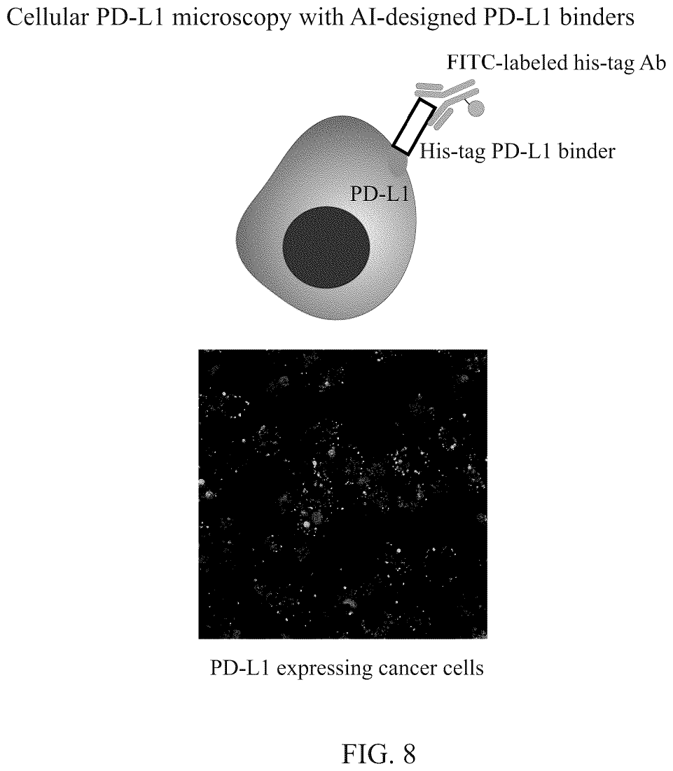

In some implementations, the computationally-designed protein binder may be used in microscopy or flow cytometry. The computationally designed protein binder may be used to detect the expression of their binding target protein in cells, or cell-derived extracellular vesicles with microscopy or flow cytometry. In some embodiments, the PD-L1 or Claudin 18.2 binding polypeptide is linked to a small molecule label. Particularly, the PD-L1 binding polypeptide is linked to the small molecule label either N-terminal or C-terminal to SEQ ID NO: 4, 5, 22, 23, or 24. In some embodiments, the small molecule label can be a fluorescent probe (e.g., organic dyes, quantum dots), which can be crosslinked to the polypeptide. In some embodiments, the small molecule label can be a biotin molecule, which is linked to the polypeptide through chemical or enzymatic biotinylation. In some embodiments, the PD-L1 or Claudin 18.2 binding polypeptide, further fused to a second polypeptide (e.g., his-tag, MBP-tag, Avi-tag, cGFP-tag) which is positioned either N-terminal or C-terminal to SEQ ID NO: 4, 5, 22, 23, or 24. In some implementations, the labeled PD-L1 binding polypeptide may be used to stain PD-L1 protein directly on the cell surface with fluorescence microscopy or flow cytometry. In some implementations, the his-tag-linked polypeptide binder may be first applied to bind a target on the cell surface, and then a fluorescence-labeled anti-his tag antibody may be applied to indirectly detect target expression (FIG. 8).

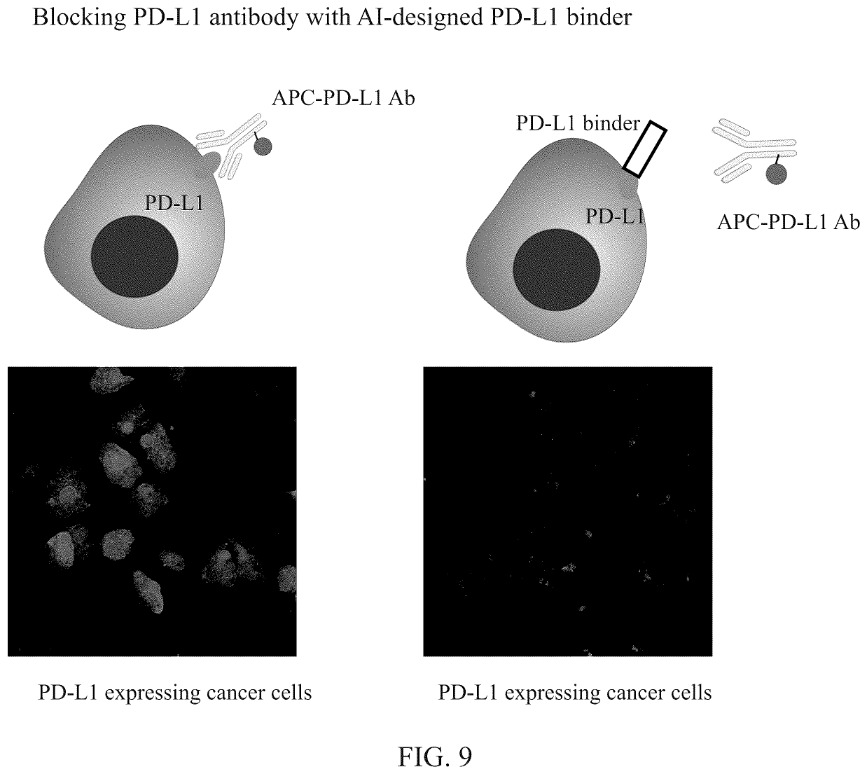

In some implementations, the computationally designed protein binder may be used as a blocking agent in cellular assays. Purified protein binder may be applied to bind its target protein on the cell surface, and block the target protein from interacting with other molecules (e.g., antibody, antibody conjugated drug, natural ligand), therefore modulating the cellular functions of target cells (FIG. 9).

### Example 1: Engineering of E. coli Vesicles for PD-L1 Binding

In some embodiments, codon-optimized genes encoding the polypeptide sequences (SEQ ID NO: 4, 5, 14, 15, 16, 17, 18, 19, 20, 21, 23, 24, 28, 30, 34, 36, 40, 42, 46, 48, 52, 54, 56, 58, 60, 62, 63, 64, and conservative mutants thereof) may be synthesized and cloned into the pET-29b (+) or pET-21b(+) E. coli plasmid expression vectors. Plasmids may be transformed into chemically competent E. coli Lemo21 cells. Bacteria may be cultured at 37° C. (or 30° C.) in cultures of lysogeny broth (LB) supplemented with 50 μg/mL of kanamycin and 30 μg/ml chloramphenicol. Cells may then be grown at 37° C. (or 30° C.) in cultures of Terrific Broth (TB) supplemented with 50 μg/mL of kanamycin, 30 μg/ml chloramphenicol, 2 mM MgSO₄, and 0.5 mM L-rhamnose, until OD600 reaches 0.4-0.6, before induction with IPTG for 12˜20 hrs.

In some embodiments, bacteria may be spun down at 4300×g for 20 mins at 4° C. The culture supernatant may be collected and filtered through a 0.45 μm vacuum filter. The filtered supernatant containing membrane vesicles may be used directly for downstream analysis, or further purified using a membrane vesicle isolation kit (e.g., SBI ExoBacteria OMV Isolation Kit). For certain downstream analysis (e.g., nanoparticle tracking analysis, RNA loading), additional buffer exchange steps may be performed so that appropriate buffers were used.

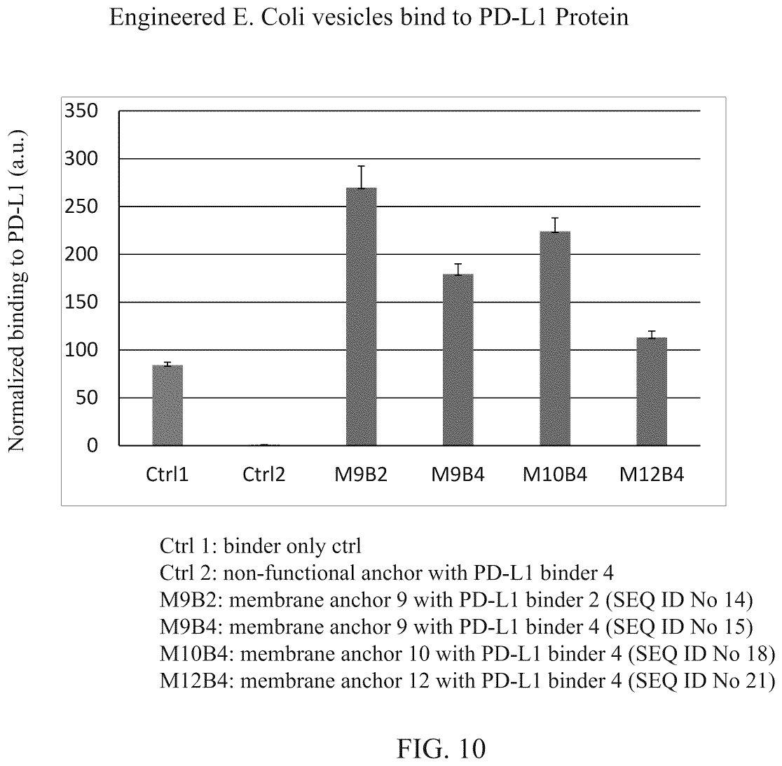

In some embodiments, clarified bacteria supernatants or purified membrane vesicles may be analyzed using the automated Magnetic electrochemical sensing assay to test binding with PD-L1. The assay may first use magnetic particles (MP) to capture bacterial membrane vesicles (EV) directly from clarified culture supernatant or from purified bacterial membrane vesicle solutions, based on the binding between PD-L1 protein on the MP and the PD-L1 binder displayed on the surface of bacteria EV. The captured EV may subsequently be labeled with an enzyme-linked detection antibody (e.g., anti-GroEL, anti-enolase, anti-OmpA, anti-his) to detect the presence of housekeeping bacteria EV marker or a marker from the designed protein scaffold. MP-EV complexes may then be magnetically concentrated on top of a sensing electrode; redox reactions and electron transfer from the electrode generated electrical current as an analytical readout (FIG. 2 and FIG. 10). The readings may be further normalized to the bacteria concentration in the cell culture. Bacteria EV that can display a functional PD-L1 binder on its surface would bind to PD-L1 and generate signals above a reference threshold; bacteria EV without a functional PD-L1 binder on its surface could not bind to PD-L1 and only generate background signals below the reference value (FIG. 10). Binding of engineered bacterial membrane vesicles to PD-L1 may also be validated with conventional immunoassays such as enzyme-linked immunosorbent assay (ELISA), or with Flow cytometry using a similar approach as described above.

In some embodiments, the sizing and quantification of the engineered vesicles may be performed on a NanoSight using Nanoparticle Tracking and Analysis software. Samples may be diluted, tested at room temperature (RT), and allowed to equilibrate prior to analysis.

### Example 2: Engineering of E. coli Vesicles for Claudin 18.2 Binding

In some embodiments, codon-optimized genes encoding the polypeptide sequences (SEQ ID NO: 22, 26, 32, 38, 44, and 50, and conservative mutants thereof) may be synthesized and cloned into the pET-29b(+) E. coli plasmid expression vectors. Plasmids may be transformed into chemically competent E. coli Lemo21 cells. Bacteria may be cultured at 37° C. (or 30° C.) in cultures of lysogeny broth (LB) supplemented with 50 μg/mL of kanamycin and 30 μg/ml chloramphenicol. Cells may then be grown at 37° C. (or 30° C.) in cultures of Terrific Broth (TB) supplemented with 50 μg/mL of kanamycin, 30 μg/ml chloramphenicol, 2 mM MgSO₄, and 0.5 mM L-rhamnose, until OD600 reaches 0.4-0.6, before induction with IPTG for 12˜20 hrs.

In some embodiments, bacteria may be spun down at 4300×g for 20 mins at 4° C. The culture supernatant may be collected and filter through a 0.45 μm vacuum filter. The filtered supernatant containing membrane vesicles may be used directly for downstream analysis, or further purified using a membrane vesicle isolation kit (e.g., SBI ExoBacteria OMV Isolation Kit). For certain downstream analysis (e.g., nanoparticle tracking analysis, RNA loading), additional buffer exchange steps may be performed so that appropriate buffers were used.

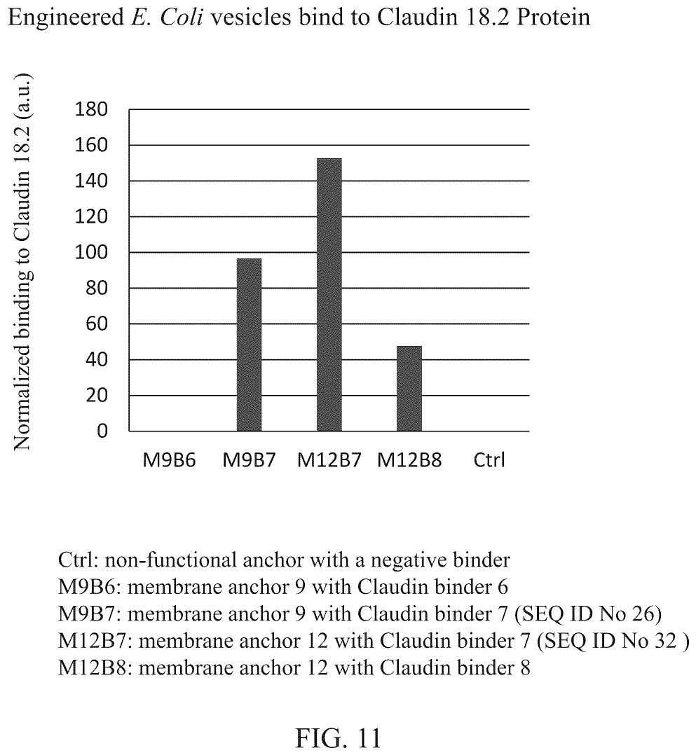

In some embodiments, clarified bacteria supernatants or purified membrane vesicles may be analyzed using the automated Magnetic electrochemical sensing assay to test binding with Claudin 18.2. The assay may first use magnetic particles (MP) to capture bacterial membrane vesicles (EV) directly from clarified culture supernatant or from purified bacterial membrane vesicle solutions, based on the binding between Claudin 18.2 protein on the MP and the Claudin 18.2 binder displayed on the surface of bacteria EV. The captured EV may subsequently be labeled with an enzyme-linked detection antibody (e.g., anti-GroEL, anti-enolase, anti-OmpA, anti-his) to detect the presence of housekeeping bacteria EV marker or a marker form the designed protein scaffold. MP-EV complexes may then be magnetically concentrated on top of a sensing electrode; redox reactions and electron transfer from the electrode generated electrical current as an analytical readout (FIG. 2 and FIG. 11). The readings may be further normalized to the bacteria concentration in the cell culture. Bacteria EV that can display a functional Claudin 18.2 binder on its surface would bind to Claudin 18.2 and generate signals above a reference threshold; bacteria EV without a functional Claudin 18.2 binder on its surface could not bind to Claudin 18.2 and only generate background signals below the reference value (FIG. 11). Binding of engineered bacterial membrane vesicles to Claudin 18.2 may also be validated with conventional immunoassays such as enzyme-linked immunosorbent assay (ELISA), or with Flow cytometry using a similar approach as described above.

In some embodiments, the sizing and quantification of the engineered vesicles may be performed on a NanoSight using Nanoparticle Tracking and Analysis software. Samples may be diluted, tested at RT, and allowed to equilibrate prior to analysis.

### Example 3: Cellular Uptake of Engineered E. coli Membrane Vesicles (EV)

In some embodiments, modified cGFP mRNAs or eGFP circRNA may be synthesized by in vitro transcription and encapsulated into the engineered E. coli EVs via electroporation. For instance, electroporation can be performed in various settings to achieve optimal encapsulation efficiency. Examples include, but are not limited to, electroporation at 0.4 k V/cm for 30 ms, 0.44 k V/cm for 30 ms, 0.53 kV/cm for 30 ms, 0.67 kV/cm for 40 ms, 0.8 kV/cm for 10 ms, 0.8 kV/cm for 25 ms, 1.2 kV/cm for 20 ms, 1.7 kV/cm for 12 ms, 2.3 kV/cm for 10 ms, 10 kV/cm for 2 ms, 21 kV/cm for 2 ms, and other suitable combinations. Each set of electroporation conditions may result in distinct loading efficiencies. The electroporation may be carried out using a commercial Invitrogen™ Neon™ Transfection System, BTX ECM 630, Bio-Rad GenePulser, or a customized electroporation device.

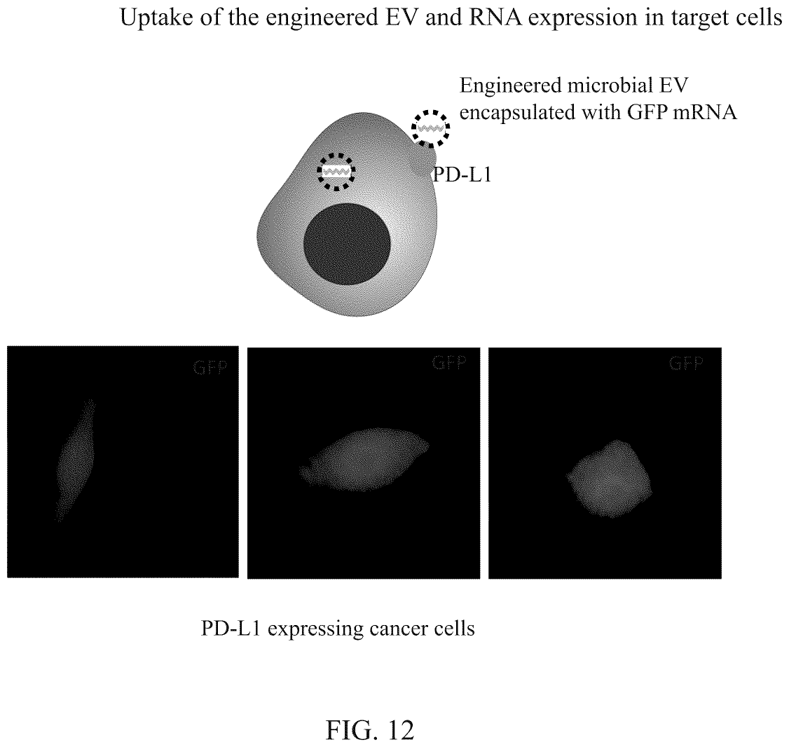

In some embodiments, human cells (e.g., RKO colon cancer cells, positive PD-L1 expression) may be cultured in 96-well plates in growth media at 37° C. cGFP RNA-encapsulated E. coli EVs may be added to the culture media of human cancer cell lines. The GFP expression in target cells may be measured by fluorescence imaging (40×, EVOS M7000) to assess EV uptake and RNA translation. 24 hr after the addition of cGFP mRNA-encapsulated E. coli EVs, expression of GFP in target RKO cells may be detected (FIG. 12). As a control, direct addition of eGFP mRNA into the cell culture does not result in GFP expression. As another control, mixing cGFP mRNA with E. coli EV without electroporation does not result in GFP expression either.

### Example 4: Cellular Uptake of Micro Algae Membrane Vesicles (EV)

In some embodiments, two wild-type microalgae strains (Spirulina, Tetraselmis Chuii) may be purchased from Algae Research Supply and cultured under their standard culture conditions. Growth media supernatants may be harvested from the algae culture and spun down first at 500 g for 5 mins at 4° C., and then at 3000 g for 20 min at 4° C. The supernatant may then be filtered through a 0.45 μm vacuum filter. The filtered supernatant containing membrane vesicles may be used directly for downstream analysis, or further purified using a membrane vesicle isolation kit (e.g., SBI ExoBacteria OMV Isolation Kit). For certain downstream analysis (e.g., nanoparticle tracking analysis, RNA loading), additional buffer exchange steps may be performed so that appropriate buffers are used.

In some embodiments, the sizing and quantification of the microalgae vesicles may be performed on a NanoSight using Nanoparticle Tracking and Analysis software. Samples may be diluted, tested at RT, and allowed to equilibrate prior to analysis (FIG. 1).

In some embodiments, modified cGFP mRNAs or cGFP circRNA may be synthesized by in vitro transcription and encapsulated into the microalgae EVs via electroporation. For instance, electroporation can be performed in various settings to achieve optimal encapsulation efficiency. Examples include, but are not limited to, electroporation at 0.4 k V/cm for 30 ms, 0.44 kV/cm for 30 ms, 0.53 kV/cm for 30 ms, 0.67 kV/cm for 40 ms, 0.8 kV/cm for 10 ms, 0.8 kV/cm for 25 ms, 1.2 kV/cm for 20 ms, 1.7 kV/cm for 12 ms, 2.3 kV/cm for 10 ms, 10 kV/cm for 2 ms, 21 kV/cm for 2 ms, and other suitable combinations. Each set of electroporation conditions may result in distinct loading efficiencies. The electroporation may be carried out using a commercial Invitrogen™ Neon™ Transfection System, BTX ECM 630, Bio-Rad GenePulser, or a customized electroporation device.

In some embodiments, human cells (e.g., RKO colon cancer cells, positive PD-L1 expression) may be cultured in 96-well plates in growth media at 37° C. cGFP RNA-encapsulated microalgae EVs may be added to the culture media of human cancer cell lines. The GFP expression in target cells may be measured by fluorescence imaging (40×, EVOS M7000) to assess EV uptake and RNA translation.

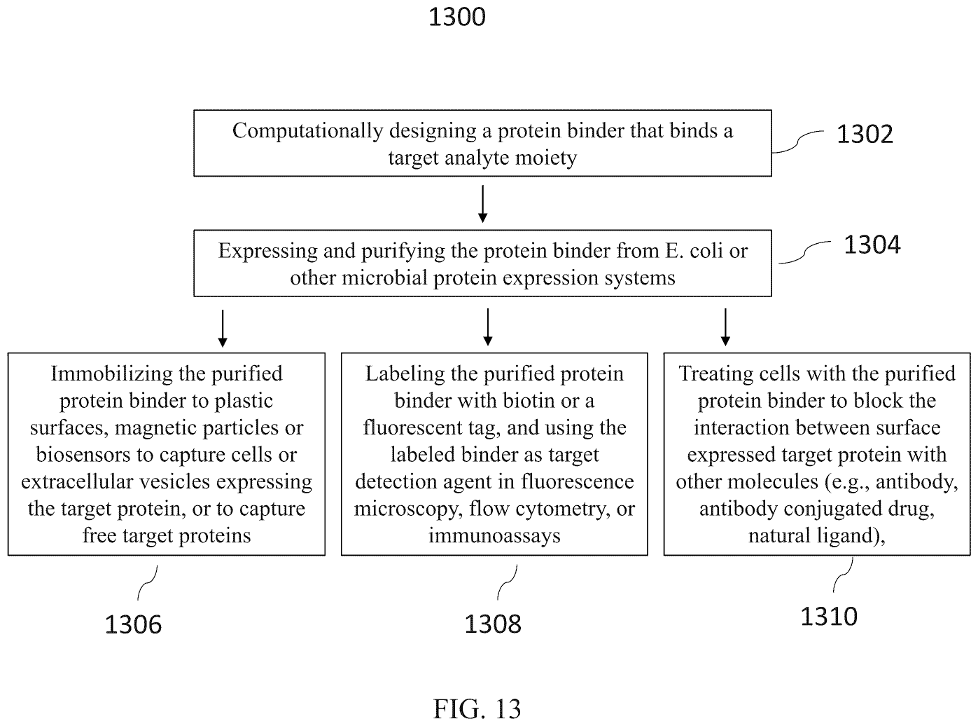

FIG. 13 is a flowchart showing a method for the design and production of protein binders and their applications in analyte capture, detection, and blocking. The method 1300 may include 1) operation 1302 computationally designing a protein binder that binds a target analyte moiety; 2) operation 1304 expressing and purifying the protein binder from E. coli or other microbial protein expression systems; and 3) performing one or more of the following: operation 1306 immobilizing the purified protein binder to plastic surfaces, magnetic particles, or biosensors to capture cells or extracellular vesicles expressing a target protein, or to capture free target proteins; operation 1308 labeling the purified protein binder with biotin or a fluorescent tag, and use the labeled protein binder as target detection agent in fluorescence microscopy, flow cytometry, or immunoassays; or operation 1310 treating cells with the purified protein binder to block the interaction between surface expressed target protein with molecules including antibodies, antibody conjugated drugs, or natural ligands.

It is to be appreciated that the Detailed Description section, and not the Summary and Abstract sections, is intended to be used to interpret the claims. The Summary and Abstract sections may set forth one or more but not all exemplary embodiments of the present disclosure as contemplated by the inventor(s), and thus, are not intended to limit the present disclosure or the appended claims in any way.

While the present disclosure has been described herein with reference to exemplary embodiments for exemplary fields and applications, it should be understood that the present disclosure is not limited thereto. Other embodiments and modifications thereto are possible and are within the scope and spirit of the present disclosure. For example, and without limiting the generality of this paragraph, embodiments are not limited to the software, hardware, firmware, and/or entities illustrated in the figures and/or described herein. Further, embodiments (whether or not explicitly described herein) have significant utility to fields and applications beyond the examples described herein.

Embodiments have been described herein with the aid of functional building blocks illustrating the implementation of specified functions and relationships thereof. The boundaries of these functional building blocks have been arbitrarily defined herein for the convenience of the description. Alternate boundaries can be defined as long as the specified functions and relationships (or equivalents thereof) are appropriately performed. Also, alternative embodiments may perform functional blocks, steps, operations, methods, etc. using orderings different than those described herein.

The breadth and scope of the present disclosure should not be limited by any of the above-described exemplary embodiments, but should be defined only in accordance with the following claims and their equivalents.

All publications, patents, and patent applications mentioned in this specification are indicative of the level of skill of those skilled in the art to which the present disclosure pertains, and are herein incorporated by reference to the same extent as if each individual publication, patent or patent application was specifically and individually indicated to be incorporated by reference.

1. An engineered delivery system, comprising:
a bacterial membrane vesicle derived from bacteria; and
one or more non-bacterial proteins for anchoring to the bacterial membrane vesicle.

2. The engineered delivery system of claim 1, wherein the one or more non-bacterial proteins comprise a mammalian membrane-associated protein or a fragment thereof.

3. The engineered delivery system of claim 1, wherein the one or more non-bacterial proteins are further linked to a synthetic polypeptide binder, either directly or by a linker.

4. The engineered delivery system of claim 3, wherein the synthetic polypeptide binder comprises a nucleic acid binding domain.

5. The engineered delivery system of claim 3, wherein the synthetic polypeptide binder comprises an amino acid sequence as set forth in SEQ ID NO: 4, 5, 22, 23, 24, or 61.

6. The engineered delivery system of claim 1, wherein the bacteria are gram-negative bacteria.

7. The engineered delivery system of claim 1, wherein the one or more non-bacterial proteins comprise mammalian protein voltage-dependent anion-selective channel 1 (VDAC1), mitochondrial carrier homolog 2 (MTCH2), or acyl-CoA synthetase long-chain family member 1 (ACSL1), or a fragment thereof.

8. The engineered delivery system of claim 7, wherein the mammalian protein comprises an amino acid sequence as set forth in SEQ ID NO: 1, 2, or 3.

9. The engineered delivery system of claim 3, wherein full amino acid sequences of the one or more non-bacterial proteins are set forth in SEQ ID NO: 6, 7, 8, 9, 10, 11, 12, 13, 25, 27, 29, 31, 33, 35, 37, 39, 41, 43, 45, 47, 49, 51, 53, 55, 57, or 59.

10. The engineered delivery system of claim 1, further comprising a nucleic acid, a protein, a complex thereof, or a combination thereof within the bacterial membrane vesicle.

11. The engineered delivery system of claim 2, further comprising a nucleic acid, a protein, a complex thereof, or a combination thereof within the bacterial membrane vesicle.

12. A synthetic polypeptide binding to programmed death-ligand 1 (PD-L1), comprising an amino acid sequence as set forth in SEQ ID NO: 4, 5, 23, or 24, including conservative mutants thereof.

13. The synthetic polypeptide of claim 12, wherein the synthetic polypeptide is linked to a small molecule label, and the small molecule label is positioned either N-terminal or C-terminal to SEQ ID NO: 4, 5, 23, or 24.

14. The synthetic polypeptide of claim 12, further comprising a second polypeptide either N-terminal or C-terminal to SEQ ID NO: 4, 5, 23, or 24.

15. The synthetic polypeptide of claim 12, further comprising a bacterial signal peptide.

16. The synthetic polypeptide of claim 12, wherein a full amino acid sequence of the synthetic polypeptide is set forth in SEQ ID NO: 14, 15, 16, 17, 18, 19, 20, 21, 28, 30, 34, 36, 40, 42, 46, 48, 52, 54, 56, 58, 60, 62, 63, or 64.

17. A synthetic polypeptide specifically binding to Claudin 18.2, comprising an amino acid sequence as set forth in SEQ ID NO: 22, including conservative mutants thereof.

18. The synthetic polypeptide of claim 17, wherein the synthetic polypeptide is further linked to a small molecule label either N-terminal or C-terminal to SEQ ID NO: 22.

19. The synthetic polypeptide of claim 17, further comprising a second polypeptide either N-terminal or C-terminal to SEQ ID NO: 22.

20. The synthetic polypeptide of claim 17, wherein a full amino acid sequence of the synthetic polypeptide is set forth in SEQ ID NO: 26, 32, 38, 44, or 50.


SEQUENCE LISTING (64 sequences)

SEQ ID NO: 1 - protein, 247 aa, Homo sapiens
GLIKLDLKTKSENGLEFTSSGSANTETTKVTGSLETKYRWTEYGLTFTEKWNTDNTLGTEITVEDQLARGLKLTFDSSFSPNTGKKNAKIKTGYKREHINLGCDMDFDIAGPSIRGALVLGYEGWLAGYQMNFETAKSRVTQSNFAVGYKTDEFQLHTNVNDGTEFGGSIYQKVNKKLETAVNLAWTAGNSNTRFGIAAKYQIDPDACFSAKVNNSSLIGLGYTQTLKPGIKLTLSALLDGKNVNAG

SEQ ID NO: 2 - protein, 302 aa, Homo sapiens
ADAASQVLLGSGLTILSQPLMYVKVLIQVGYEPLPPTIGRNIFGRQVCQLPGLFSYAQHIASIDGRRGLFTGLTPRLCSGVLGTVVHGKVLQHYQESDKGEELGPGNVQKEVSSSFDHVIKETTREMIARSAATLITHPFHVITLRSMVQFIGRESKYCGLCDSIITIYREEGILGFFAGLVPRLLGDILSLWLCNSLAYLVNTYALDSGVSTMNEMKSYSQAVTGFFASMLTYPFVLVSNLMAVNNCGLAGGCPPYSPIYTSWIDCWCMLQKEGNMSRGNSLFFRKVPFGKTYCCDLKMLI

SEQ ID NO: 3 - protein, 44 aa, Homo sapiens
QAHELFRYFRMPELVDFRQYVRTLPTNTLMGFGAFAALTTFWYA

SEQ ID NO: 4 - protein, 100 aa, synthetic construct
GTVTLTVTVTATKDGEIKVSVMVRSSNKDVTLSISVSGGTLLNSSTATGSSGVAIGGATVYLKAGQTTTVTATATNVSADATVTASVGGDATFSSSTSTA

SEQ ID NO: 5 - protein, 110 aa, synthetic construct
DLEEFFEECLKEAEEVRKEVLEGKARIQIKESVRKEDPEGGTYVCGVISLGPKGGKFHVVFHKEDPEGKEETLARFEFKVEGGKVELLELKSKDPELKKLAEKYAEKLLE

SEQ ID NO: 6 - protein, 347 aa, synthetic construct
GLIKLDLKTKSENGLEFTSSGSANTETTKVTGSLETKYRWTEYGLTFTEKWNTDNTLGTEITVEDQLARGLKLTFDSSFSPNTGKKNAKIKTGYKREHINLGCDMDFDIAGPSIRGALVLGYEGWLAGYQMNFETAKSRVTQSNFAVGYKTDEFQLHTNVNDGTEFGGSIYQKVNKKLETAVNLAWTAGNSNTRFGIAAKYQIDPDACFSAKVNNSSLIGLGYTQTLKPGIKLTLSALLDGKNVNAGGTVTLTVTVTATKDGEIKVSVMVRSSNKDVTLSISVSGGTLLNSSTATGSSGVAIGGATVYLKAGQTTTVTATATNVSADATVTASVGGDATFSSSTSTA

SEQ ID NO: 7 - protein, 357 aa, synthetic construct
GLIKLDLKTKSENGLEFTSSGSANTETTKVTGSLETKYRWTEYGLTFTEKWNTDNTLGTEITVEDQLARGLKLTFDSSFSPNTGKKNAKIKTGYKREHINLGCDMDFDIAGPSIRGALVLGYEGWLAGYQMNFETAKSRVTQSNFAVGYKTDEFQLHTNVNDGTEFGGSIYQKVNKKLETAVNLAWTAGNSNTRFGIAAKYQIDPDACFSAKVNNSSLIGLGYTQTLKPGIKLTLSALLDGKNVNAGDLEEFFEECLKEAEEVRKEVLEGKARIQIKESVRKEDPEGGTYVCGVISLGPKGGKFHVVFHKEDPEGKEETLARFEFKVEGGKVELLELKSKDPELKKLAEKYAEKLLE

SEQ ID NO: 8 - protein, 407 aa, synthetic construct
GTVTLTVTVTATKDGEIKVSVMVRSSNKDVTLSISVSGGTLLNSSTATGSSGVAIGGATVYLKAGQTTTVTATATNVSADATVTASVGGDATFSSSTSTAGGGGSADAASQVLLGSGLTILSQPLMYVKVLIQVGYEPLPPTIGRNIFGRQVCQLPGLFSYAQHIASIDGRRGLFTGLTPRLCSGVLGTVVHGKVLQHYQESDKGEELGPGNVQKEVSSSFDHVIKETTREMIARSAATLITHPFHVITLRSMVQFIGRESKYCGLCDSIITIYREEGILGFFAGLVPRLLGDILSLWLCNSLAYLVNTYALDSGVSTMNEMKSYSQAVTGFFASMLTYPFVLVSNLMAVNNCGLAGGCPPYSPIYTSWIDCWCMLQKEGNMSRGNSLFFRKVPFGKTYCCDLKMLI

SEQ ID NO: 9 - protein, 407 aa, synthetic construct
ADAASQVLLGSGLTILSQPLMYVKVLIQVGYEPLPPTIGRNIFGRQVCQLPGLFSYAQHIASIDGRRGLFTGLTPRLCSGVLGTVVHGKVLQHYQESDKGEELGPGNVQKEVSSSFDHVIKETTREMIARSAATLITHPFHVITLRSMVQFIGRESKYCGLCDSIITIYREEGILGFFAGLVPRLLGDILSLWLCNSLAYLVNTYALDSGVSTMNEMKSYSQAVTGFFASMLTYPFVLVSNLMAVNNCGLAGGCPPYSPIYTSWIDCWCMLQKEGNMSRGNSLFFRKVPFGKTYCCDLKMLIGGGGSGTVTLTVTVTATKDGEIKVSVMVRSSNKDVTLSISVSGGTLLNSSTATGSSGVAIGGATVYLKAGQTTTVTATATNVSADATVTASVGGDATFSSSTSTA

SEQ ID NO: 10 - protein, 417 aa, synthetic construct
DLEEFFEECLKEAEEVRKEVLEGKARIQIKESVRKEDPEGGTYVCGVISLGPKGGKFHVVFHKEDPEGKEETLARFEFKVEGGKVELLELKSKDPELKKLAEKYAEKLLEGGGGSADAASQVLLGSGLTILSQPLMYVKVLIQVGYEPLPPTIGRNIFGRQVCQLPGLFSYAQHIASIDGRRGLFTGLTPRLCSGVLGTVVHGKVLQHYQESDKGEELGPGNVQKEVSSSFDHVIKETTREMIARSAATLITHPFHVITLRSMVQFIGRESKYCGLCDSIITIYREEGILGFFAGLVPRLLGDILSLWLCNSLAYLVNTYALDSGVSTMNEMKSYSQAVTGFFASMLTYPFVLVSNLMAVNNCGLAGGCPPYSPIYTSWIDCWCMLQKEGNMSRGNSLFFRKVPFGKTYCCDLKMLI

SEQ ID NO: 11 - protein, 149 aa, synthetic construct
GTVTLTVTVTATKDGEIKVSVMVRSSNKDVTLSISVSGGTLLNSSTATGSSGVAIGGATVYLKAGQTTTVTATATNVSADATVTASVGGDATFSSSTSTAGGGGSQAHELFRYFRMPELVDFRQYVRTLPTNTLMGFGAFAALTTFWYA

SEQ ID NO: 12 - protein, 149 aa, synthetic construct
QAHELFRYFRMPELVDFRQYVRTLPTNTLMGFGAFAALTTFWYAGGGGSGTVTLTVTVTATKDGEIKVSVMVRSSNKDVTLSISVSGGTLLNSSTATGSSGVAIGGATVYLKAGQTTTVTATATNVSADATVTASVGGDATFSSSTSTA

SEQ ID NO: 13 - protein, 159 aa, synthetic construct
QAHELFRYFRMPELVDFRQYVRTLPTNTLMGFGAFAALTTFWYAGGGGSDLEEFFEECLKEAEEVRKEVLEGKARIQIKESVRKEDPEGGTYVCGVISLGPKGGKFHVVFHKEDPEGKEETLARFEFKVEGGKVELLELKSKDPELKKLAEKYAEKLLE

SEQ ID NO: 14 - protein, 366 aa, synthetic construct
MKKIWLALAGLVLAFSASAGLIKLDLKTKSENGLEFTSSGSANTETTKVTGSLETKYRWTEYGLTFTEKWNTDNTLGTEITVEDQLARGLKLTFDSSFSPNTGKKNAKIKTGYKREHINLGCDMDFDIAGPSIRGALVLGYEGWLAGYQMNFETAKSRVTQSNFAVGYKTDEFQLHTNVNDGTEFGGSIYQKVNKKLETAVNLAWTAGNSNTRFGIAAKYQIDPDACFSAKVNNSSLIGLGYTQTLKPGIKLTLSALLDGKNVNAGGTVTLTVTVTATKDGEIKVSVMVRSSNKDVTLSISVSGGTLLNSSTATGSSGVAIGGATVYLKAGQTTTVTATATNVSADATVTASVGGDATFSSSTSTA

SEQ ID NO: 15 - protein, 376 aa, synthetic construct
MKKIWLALAGLVLAFSASAGLIKLDLKTKSENGLEFTSSGSANTETTKVTGSLETKYRWTEYGLTFTEKWNTDNTLGTEITVEDQLARGLKLTFDSSFSPNTGKKNAKIKTGYKREHINLGCDMDFDIAGPSIRGALVLGYEGWLAGYQMNFETAKSRVTQSNFAVGYKTDEFQLHTNVNDGTEFGGSIYQKVNKKLETAVNLAWTAGNSNTRFGIAAKYQIDPDACFSAKVNNSSLIGLGYTQTLKPGIKLTLSALLDGKNVNAGDLEEFFEECLKEAEEVRKEVLEGKARIQIKESVRKEDPEGGTYVCGVISLGPKGGKFHVVFHKEDPEGKEETLARFEFKVEGGKVELLELKSKDPELKKLAEKYAEKLLE

SEQ ID NO: 16 - protein, 426 aa, synthetic construct
MKKIWLALAGLVLAFSASAGTVTLTVTVTATKDGEIKVSVMVRSSNKDVTLSISVSGGTLLNSSTATGSSGVAIGGATVYLKAGQTTTVTATATNVSADATVTASVGGDATFSSSTSTAGGGGSADAASQVLLGSGLTILSQPLMYVKVLIQVGYEPLPPTIGRNIFGRQVCQLPGLFSYAQHIASIDGRRGLFTGLTPRLCSGVLGTVVHGKVLQHYQESDKGEELGPGNVQKEVSSSFDHVIKETTREMIARSAATLITHPFHVITLRSMVQFIGRESKYCGLCDSIITIYREEGILGFFAGLVPRLLGDILSLWLCNSLAYLVNTYALDSGVSTMNEMKSYSQAVTGFFASMLTYPFVLVSNLMAVNNCGLAGGCPPYSPIYTSWIDCWCMLQKEGNMSRGNSLFFRKVPFGKTYCCDLKMLI

SEQ ID NO: 17 - protein, 426 aa, synthetic construct
MKKIWLALAGLVLAFSASAADAASQVLLGSGLTILSQPLMYVKVLIQVGYEPLPPTIGRNIFGRQVCQLPGLFSYAQHIASIDGRRGLFTGLTPRLCSGVLGTVVHGKVLQHYQESDKGEELGPGNVQKEVSSSFDHVIKETTREMIARSAATLITHPFHVITLRSMVQFIGRESKYCGLCDSIITIYREEGILGFFAGLVPRLLGDILSLWLCNSLAYLVNTYALDSGVSTMNEMKSYSQAVTGFFASMLTYPFVLVSNLMAVNNCGLAGGCPPYSPIYTSWIDCWCMLQKEGNMSRGNSLFFRKVPFGKTYCCDLKMLIGGGGSGTVTLTVTVTATKDGEIKVSVMVRSSNKDVTLSISVSGGTLLNSSTATGSSGVAIGGATVYLKAGQTTTVTATATNVSADATVTASVGGDATFSSSTSTA

SEQ ID NO: 18 - protein, 436 aa, synthetic construct
MKKIWLALAGLVLAFSASADLEEFFEECLKEAEEVRKEVLEGKARIQIKESVRKEDPEGGTYVCGVISLGPKGGKFHVVFHKEDPEGKEETLARFEFKVEGGKVELLELKSKDPELKKLAEKYAEKLLEGGGGSADAASQVLLGSGLTILSQPLMYVKVLIQVGYEPLPPTIGRNIFGRQVCQLPGLFSYAQHIASIDGRRGLFTGLTPRLCSGVLGTVVHGKVLQHYQESDKGEELGPGNVQKEVSSSFDHVIKETTREMIARSAATLITHPFHVITLRSMVQFIGRESKYCGLCDSIITIYREEGILGFFAGLVPRLLGDILSLWLCNSLAYLVNTYALDSGVSTMNEMKSYSQAVTGFFASMLTYPFVLVSNLMAVNNCGLAGGCPPYSPIYTSWIDCWCMLQKEGNMSRGNSLFFRKVPFGKTYCCDLKMLI

SEQ ID NO: 19 - protein, 168 aa, synthetic construct
MKKIWLALAGLVLAFSASAGTVTLTVTVTATKDGEIKVSVMVRSSNKDVTLSISVSGGTLLNSSTATGSSGVAIGGATVYLKAGQTTTVTATATNVSADATVTASVGGDATFSSSTSTAGGGGSQAHELFRYFRMPELVDFRQYVRTLPTNTLMGFGAFAALTTFWYA

SEQ ID NO: 20 - protein, 168 aa, synthetic construct
MKKIWLALAGLVLAFSASAQAHELFRYFRMPELVDFRQYVRTLPTNTLMGFGAFAALTTFWYAGGGGSGTVTLTVTVTATKDGEIKVSVMVRSSNKDVTLSISVSGGTLLNSSTATGSSGVAIGGATVYLKAGQTTTVTATATNVSADATVTASVGGDATFSSSTSTA

SEQ ID NO: 21 - protein, 189 aa, synthetic construct
MKKIWLALAGLVLAFSASAHHHHHHGGGGSQAHELFRYFRMPELVDFRQYVRTLPTNTLMGFGAFAALTTFWYAGGGGSDLEEFFEECLKEAEEVRKEVLEGKARIQIKESVRKEDPEGGTYVCGVISLGPKGGKFHVVFHKEDPEGKEETLARFEFKVEGGKVELLELKSKDPELKKLAEKYAEKLLE

SEQ ID NO: 22 - protein, 88 aa, synthetic construct
SAALLRFLADLRLGAAAIFEGAEVGLLAEEGDPEVLAAQTREALRRAAGARRVVVLSAASPEGAALIAAVVAETAPLVGEVTFETLEA

SEQ ID NO: 23 - protein, 81 aa, synthetic construct
EAEELLKKALEYFKKAAELAKKGNKEEAEKYAIEGAKLLGKAIGLDEEGIKTLQRAAKNAAKAGTVGKYAEAMAKNVEALL

SEQ ID NO: 24 - protein, 97 aa, synthetic construct
AFEAAAARLEAEARALLARARARGPGRLYLVLQRELPSGTLETLASGDLERESLEETLARFRAALAAAPAGTYRAAVISLEPRLEVLESPPVEVEAP

SEQ ID NO: 25 - protein, 350 aa, synthetic construct
GLIKLDLKTKSENGLEFTSSGSANTETTKVTGSLETKYRWTEYGLTFTEKWNTDNTLGTEITVEDQLARGLKLTFDSSFSPNTGKKNAKIKTGYKREHINLGCDMDFDIAGPSIRGALVLGYEGWLAGYQMNFETAKSRVTQSNFAVGYKTDEFQLHTNVNDGTEFGGSIYQKVNKKLETAVNLAWTAGNSNTRFGIAAKYQIDPDACFSAKVNNSSLIGLGYTQTLKPGIKLTLSALLDGKNVNAGGGGGSGGGGSGGGGSSAALLRFLADLRLGAAAIFEGAEVGLLAEEGDPEVLAAQTREALRRAAGARRVVVLSAASPEGAALIAAVVAETAPLVGEVTFETLEA

SEQ ID NO: 26 - protein, 369 aa, synthetic construct
MKKIWLALAGLVLAFSASAGLIKLDLKTKSENGLEFTSSGSANTETTKVTGSLETKYRWTEYGLTFTEKWNTDNTLGTEITVEDQLARGLKLTFDSSFSPNTGKKNAKIKTGYKREHINLGCDMDFDIAGPSIRGALVLGYEGWLAGYQMNFETAKSRVTQSNFAVGYKTDEFQLHTNVNDGTEFGGSIYQKVNKKLETAVNLAWTAGNSNTRFGIAAKYQIDPDACFSAKVNNSSLIGLGYTQTLKPGIKLTLSALLDGKNVNAGGGGGSGGGGSGGGGSSAALLRFLADLRLGAAAIFEGAEVGLLAEEGDPEVLAAQTREALRRAAGARRVVVLSAASPEGAALIAAVVAETAPLVGEVTFETLEA

SEQ ID NO: 27 - protein, 328 aa, synthetic construct
GLIKLDLKTKSENGLEFTSSGSANTETTKVTGSLETKYRWTEYGLTFTEKWNTDNTLGTEITVEDQLARGLKLTFDSSFSPNTGKKNAKIKTGYKREHINLGCDMDFDIAGPSIRGALVLGYEGWLAGYQMNFETAKSRVTQSNFAVGYKTDEFQLHTNVNDGTEFGGSIYQKVNKKLETAVNLAWTAGNSNTRFGIAAKYQIDPDACFSAKVNNSSLIGLGYTQTLKPGIKLTLSALLDGKNVNAGEAEELLKKALEYFKKAAELAKKGNKEEAEKYAIEGAKLLGKAIGLDEEGIKTLQRAAKNAAKAGTVGKYAEAMAKNVEALL

SEQ ID NO: 28 - protein, 347 aa, synthetic construct
MKKIWLALAGLVLAFSASAGLIKLDLKTKSENGLEFTSSGSANTETTKVTGSLETKYRWTEYGLTFTEKWNTDNTLGTEITVEDQLARGLKLTFDSSFSPNTGKKNAKIKTGYKREHINLGCDMDFDIAGPSIRGALVLGYEGWLAGYQMNFETAKSRVTQSNFAVGYKTDEFQLHTNVNDGTEFGGSIYQKVNKKLETAVNLAWTAGNSNTRFGIAAKYQIDPDACFSAKVNNSSLIGLGYTQTLKPGIKLTLSALLDGKNVNAGEAEELLKKALEYFKKAAELAKKGNKEEAEKYAIEGAKLLGKAIGLDEEGIKTLQRAAKNAAKAGTVGKYAEAMAKNVEALL

SEQ ID NO: 29 - protein, 344 aa, synthetic construct
GLIKLDLKTKSENGLEFTSSGSANTETTKVTGSLETKYRWTEYGLTFTEKWNTDNTLGTEITVEDQLARGLKLTFDSSFSPNTGKKNAKIKTGYKREHINLGCDMDFDIAGPSIRGALVLGYEGWLAGYQMNFETAKSRVTQSNFAVGYKTDEFQLHTNVNDGTEFGGSIYQKVNKKLETAVNLAWTAGNSNTRFGIAAKYQIDPDACFSAKVNNSSLIGLGYTQTLKPGIKLTLSALLDGKNVNAGAFEAAAARLEAEARALLARARARGPGRLYLVLQRELPSGTLETLASGDLERESLEETLARFRAALAAAPAGTYRAAVISLEPRLEVLESPPVEVEAP

SEQ ID NO: 30 - protein, 363 aa, synthetic construct
MKKIWLALAGLVLAFSASAGLIKLDLKTKSENGLEFTSSGSANTETTKVTGSLETKYRWTEYGLTFTEKWNTDNTLGTEITVEDQLARGLKLTFDSSFSPNTGKKNAKIKTGYKREHINLGCDMDFDIAGPSIRGALVLGYEGWLAGYQMNFETAKSRVTQSNFAVGYKTDEFQLHTNVNDGTEFGGSIYQKVNKKLETAVNLAWTAGNSNTRFGIAAKYQIDPDACFSAKVNNSSLIGLGYTQTLKPGIKLTLSALLDGKNVNAGAFEAAAARLEAEARALLARARARGPGRLYLVLQRELPSGTLETLASGDLERESLEETLARFRAALAAAPAGTYRAAVISLEPRLEVLESPPVEVEAP

SEQ ID NO: 31 - protein, 137 aa, synthetic construct
SAALLRFLADLRLGAAAIFEGAEVGLLAEEGDPEVLAAQTREALRRAAGARRVVVLSAASPEGAALIAAVVAETAPLVGEVTFETLEAGGGGSQAHELFRYFRMPELVDFRQYVRTLPTNTLMGFGAFAALTTFWYA

SEQ ID NO: 32 - protein, 156 aa, synthetic construct
MKKIWLALAGLVLAFSASASAALLRFLADLRLGAAAIFEGAEVGLLAEEGDPEVLAAQTREALRRAAGARRVVVLSAASPEGAALIAAVVAETAPLVGEVTFETLEAGGGGSQAHELFRYFRMPELVDFRQYVRTLPTNTLMGFGAFAALTTFWYA

SEQ ID NO: 33 - protein, 130 aa, synthetic construct
EAEELLKKALEYFKKAAELAKKGNKEEAEKYAIEGAKLLGKAIGLDEEGIKTLQRAAKNAAKAGTVGKYAEAMAKNVEALLGGGGSQAHELFRYFRMPELVDFRQYVRTLPTNTLMGFGAFAALTTFWYA

SEQ ID NO: 34 - protein, 149 aa, synthetic construct
MKKIWLALAGLVLAFSASAEAEELLKKALEYFKKAAELAKKGNKEEAEKYAIEGAKLLGKAIGLDEEGIKTLQRAAKNAAKAGTVGKYAEAMAKNVEALLGGGGSQAHELFRYFRMPELVDFRQYVRTLPTNTLMGFGAFAALTTFWYA

SEQ ID NO: 35 - protein, 146 aa, synthetic construct
AFEAAAARLEAEARALLARARARGPGRLYLVLQRELPSGTLETLASGDLERESLEETLARFRAALAAAPAGTYRAAVISLEPRLEVLESPPVEVEAPGGGGSQAHELFRYFRMPELVDFRQYVRTLPTNTLMGFGAFAALTTFWYA

SEQ ID NO: 36 - protein, 165 aa, synthetic construct
MKKIWLALAGLVLAFSASAAFEAAAARLEAEARALLARARARGPGRLYLVLQRELPSGTLETLASGDLERESLEETLARFRAALAAAPAGTYRAAVISLEPRLEVLESPPVEVEAPGGGGSQAHELFRYFRMPELVDFRQYVRTLPTNTLMGFGAFAALTTFWYA

SEQ ID NO: 37 - protein, 137 aa, synthetic construct
QAHELFRYFRMPELVDFRQYVRTLPTNTLMGFGAFAALTTFWYAGGGGSSAALLRFLADLRLGAAAIFEGAEVGLLAEEGDPEVLAAQTREALRRAAGARRVVVLSAASPEGAALIAAVVAETAPLVGEVTFETLEA

SEQ ID NO: 38 - protein, 156 aa, synthetic construct
MKKIWLALAGLVLAFSASAQAHELFRYFRMPELVDFRQYVRTLPTNTLMGFGAFAALTTFWYAGGGGSSAALLRFLADLRLGAAAIFEGAEVGLLAEEGDPEVLAAQTREALRRAAGARRVVVLSAASPEGAALIAAVVAETAPLVGEVTFETLEA

SEQ ID NO: 39 - protein, 130 aa, synthetic construct
QAHELFRYFRMPELVDFRQYVRTLPTNTLMGFGAFAALTTFWYAGGGGSEAEELLKKALEYFKKAAELAKKGNKEEAEKYAIEGAKLLGKAIGLDEEGIKTLQRAAKNAAKAGTVGKYAEAMAKNVEALL

SEQ ID NO: 40 - protein, 149 aa, synthetic construct
MKKIWLALAGLVLAFSASAQAHELFRYFRMPELVDFRQYVRTLPTNTLMGFGAFAALTTFWYAGGGGSEAEELLKKALEYFKKAAELAKKGNKEEAEKYAIEGAKLLGKAIGLDEEGIKTLQRAAKNAAKAGTVGKYAEAMAKNVEALL

SEQ ID NO: 41 - protein, 146 aa, synthetic construct
QAHELFRYFRMPELVDFRQYVRTLPTNTLMGFGAFAALTTFWYAGGGGSAFEAAAARLEAEARALLARARARGPGRLYLVLQRELPSGTLETLASGDLERESLEETLARFRAALAAAPAGTYRAAVISLEPRLEVLESPPVEVEAP

SEQ ID NO: 42 - protein, 165 aa, synthetic construct
MKKIWLALAGLVLAFSASAQAHELFRYFRMPELVDFRQYVRTLPTNTLMGFGAFAALTTFWYAGGGGSAFEAAAARLEAEARALLARARARGPGRLYLVLQRELPSGTLETLASGDLERESLEETLARFRAALAAAPAGTYRAAVISLEPRLEVLESPPVEVEAP

SEQ ID NO: 43 - protein, 395 aa, synthetic construct
SAALLRFLADLRLGAAAIFEGAEVGLLAEEGDPEVLAAQTREALRRAAGARRVVVLSAASPEGAALIAAVVAETAPLVGEVTFETLEAGGGGSADAASQVLLGSGLTILSQPLMYVKVLIQVGYEPLPPTIGRNIFGRQVCQLPGLFSYAQHIASIDGRRGLFTGLTPRLCSGVLGTVVHGKVLQHYQESDKGEELGPGNVQKEVSSSFDHVIKETTREMIARSAATLITHPFHVITLRSMVQFIGRESKYCGLCDSIITIYREEGILGFFAGLVPRLLGDILSLWLCNSLAYLVNTYALDSGVSTMNEMKSYSQAVTGFFASMLTYPFVLVSNLMAVNNCGLAGGCPPYSPIYTSWIDCWCMLQKEGNMSRGNSLFFRKVPFGKTYCCDLKMLI

SEQ ID NO: 44 - protein, 414 aa, synthetic construct
MKKIWLALAGLVLAFSASASAALLRFLADLRLGAAAIFEGAEVGLLAEEGDPEVLAAQTREALRRAAGARRVVVLSAASPEGAALIAAVVAETAPLVGEVTFETLEAGGGGSADAASQVLLGSGLTILSQPLMYVKVLIQVGYEPLPPTIGRNIFGRQVCQLPGLFSYAQHIASIDGRRGLFTGLTPRLCSGVLGTVVHGKVLQHYQESDKGEELGPGNVQKEVSSSFDHVIKETTREMIARSAATLITHPFHVITLRSMVQFIGRESKYCGLCDSIITIYREEGILGFFAGLVPRLLGDILSLWLCNSLAYLVNTYALDSGVSTMNEMKSYSQAVTGFFASMLTYPFVLVSNLMAVNNCGLAGGCPPYSPIYTSWIDCWCMLQKEGNMSRGNSLFFRKVPFGKTYCCDLKMLI

SEQ ID NO: 45 - protein, 388 aa, synthetic construct
EAEELLKKALEYFKKAAELAKKGNKEEAEKYAIEGAKLLGKAIGLDEEGIKTLQRAAKNAAKAGTVGKYAEAMAKNVEALLGGGGSADAASQVLLGSGLTILSQPLMYVKVLIQVGYEPLPPTIGRNIFGRQVCQLPGLFSYAQHIASIDGRRGLFTGLTPRLCSGVLGTVVHGKVLQHYQESDKGEELGPGNVQKEVSSSFDHVIKETTREMIARSAATLITHPFHVITLRSMVQFIGRESKYCGLCDSIITIYREEGILGFFAGLVPRLLGDILSLWLCNSLAYLVNTYALDSGVSTMNEMKSYSQAVTGFFASMLTYPFVLVSNLMAVNNCGLAGGCPPYSPIYTSWIDCWCMLQKEGNMSRGNSLFFRKVPFGKTYCCDLKMLI

SEQ ID NO: 46 - protein, 407 aa, synthetic construct
MKKIWLALAGLVLAFSASAEAEELLKKALEYFKKAAELAKKGNKEEAEKYAIEGAKLLGKAIGLDEEGIKTLQRAAKNAAKAGTVGKYAEAMAKNVEALLGGGGSADAASQVLLGSGLTILSQPLMYVKVLIQVGYEPLPPTIGRNIFGRQVCQLPGLFSYAQHIASIDGRRGLFTGLTPRLCSGVLGTVVHGKVLQHYQESDKGEELGPGNVQKEVSSSFDHVIKETTREMIARSAATLITHPFHVITLRSMVQFIGRESKYCGLCDSIITIYREEGILGFFAGLVPRLLGDILSLWLCNSLAYLVNTYALDSGVSTMNEMKSYSQAVTGFFASMLTYPFVLVSNLMAVNNCGLAGGCPPYSPIYTSWIDCWCMLQKEGNMSRGNSLFFRKVPFGKTYCCDLKMLI

SEQ ID NO: 47 - protein, 404 aa, synthetic construct
AFEAAAARLEAEARALLARARARGPGRLYLVLQRELPSGTLETLASGDLERESLEETLARFRAALAAAPAGTYRAAVISLEPRLEVLESPPVEVEAPGGGGSADAASQVLLGSGLTILSQPLMYVKVLIQVGYEPLPPTIGRNIFGRQVCQLPGLFSYAQHIASIDGRRGLFTGLTPRLCSGVLGTVVHGKVLQHYQESDKGEELGPGNVQKEVSSSFDHVIKETTREMIARSAATLITHPFHVITLRSMVQFIGRESKYCGLCDSIITIYREEGILGFFAGLVPRLLGDILSLWLCNSLAYLVNTYALDSGVSTMNEMKSYSQAVTGFFASMLTYPFVLVSNLMAVNNCGLAGGCPPYSPIYTSWIDCWCMLQKEGNMSRGNSLFFRKVPFGKTYCCDLKMLI

SEQ ID NO: 48 - protein, 423 aa, synthetic construct
MKKIWLALAGLVLAFSASAAFEAAAARLEAEARALLARARARGPGRLYLVLQRELPSGTLETLASGDLERESLEETLARFRAALAAAPAGTYRAAVISLEPRLEVLESPPVEVEAPGGGGSADAASQVLLGSGLTILSQPLMYVKVLIQVGYEPLPPTIGRNIFGRQVCQLPGLFSYAQHIASIDGRRGLFTGLTPRLCSGVLGTVVHGKVLQHYQESDKGEELGPGNVQKEVSSSFDHVIKETTREMIARSAATLITHPFHVITLRSMVQFIGRESKYCGLCDSIITIYREEGILGFFAGLVPRLLGDILSLWLCNSLAYLVNTYALDSGVSTMNEMKSYSQAVTGFFASMLTYPFVLVSNLMAVNNCGLAGGCPPYSPIYTSWIDCWCMLQKEGNMSRGNSLFFRKVPFGKTYCCDLKMLI

SEQ ID NO: 49 - protein, 395 aa, synthetic construct
ADAASQVLLGSGLTILSQPLMYVKVLIQVGYEPLPPTIGRNIFGRQVCQLPGLFSYAQHIASIDGRRGLFTGLTPRLCSGVLGTVVHGKVLQHYQESDKGEELGPGNVQKEVSSSFDHVIKETTREMIARSAATLITHPFHVITLRSMVQFIGRESKYCGLCDSIITIYREEGILGFFAGLVPRLLGDILSLWLCNSLAYLVNTYALDSGVSTMNEMKSYSQAVTGFFASMLTYPFVLVSNLMAVNNCGLAGGCPPYSPIYTSWIDCWCMLQKEGNMSRGNSLFFRKVPFGKTYCCDLKMLIGGGGSSAALLRFLADLRLGAAAIFEGAEVGLLAEEGDPEVLAAQTREALRRAAGARRVVVLSAASPEGAALIAAVVAETAPLVGEVTFETLEA

SEQ ID NO: 50 - protein, 414 aa, synthetic construct
MKKIWLALAGLVLAFSASAADAASQVLLGSGLTILSQPLMYVKVLIQVGYEPLPPTIGRNIFGRQVCQLPGLFSYAQHIASIDGRRGLFTGLTPRLCSGVLGTVVHGKVLQHYQESDKGEELGPGNVQKEVSSSFDHVIKETTREMIARSAATLITHPFHVITLRSMVQFIGRESKYCGLCDSIITIYREEGILGFFAGLVPRLLGDILSLWLCNSLAYLVNTYALDSGVSTMNEMKSYSQAVTGFFASMLTYPFVLVSNLMAVNNCGLAGGCPPYSPIYTSWIDCWCMLQKEGNMSRGNSLFFRKVPFGKTYCCDLKMLIGGGGSSAALLRFLADLRLGAAAIFEGAEVGLLAEEGDPEVLAAQTREALRRAAGARRVVVLSAASPEGAALIAAVVAETAPLVGEVTFETLEA

SEQ ID NO: 51 - protein, 388 aa, synthetic construct
ADAASQVLLGSGLTILSQPLMYVKVLIQVGYEPLPPTIGRNIFGRQVCQLPGLFSYAQHIASIDGRRGLFTGLTPRLCSGVLGTVVHGKVLQHYQESDKGEELGPGNVQKEVSSSFDHVIKETTREMIARSAATLITHPFHVITLRSMVQFIGRESKYCGLCDSIITIYREEGILGFFAGLVPRLLGDILSLWLCNSLAYLVNTYALDSGVSTMNEMKSYSQAVTGFFASMLTYPFVLVSNLMAVNNCGLAGGCPPYSPIYTSWIDCWCMLQKEGNMSRGNSLFFRKVPFGKTYCCDLKMLIGGGGSEAEELLKKALEYFKKAAELAKKGNKEEAEKYAIEGAKLLGKAIGLDEEGIKTLQRAAKNAAKAGTVGKYAEAMAKNVEALL

SEQ ID NO: 52 - protein, 407 aa, synthetic construct
MKKIWLALAGLVLAFSASAADAASQVLLGSGLTILSQPLMYVKVLIQVGYEPLPPTIGRNIFGRQVCQLPGLFSYAQHIASIDGRRGLFTGLTPRLCSGVLGTVVHGKVLQHYQESDKGEELGPGNVQKEVSSSFDHVIKETTREMIARSAATLITHPFHVITLRSMVQFIGRESKYCGLCDSIITIYREEGILGFFAGLVPRLLGDILSLWLCNSLAYLVNTYALDSGVSTMNEMKSYSQAVTGFFASMLTYPFVLVSNLMAVNNCGLAGGCPPYSPIYTSWIDCWCMLQKEGNMSRGNSLFFRKVPFGKTYCCDLKMLIGGGGSEAEELLKKALEYFKKAAELAKKGNKEEAEKYAIEGAKLLGKAIGLDEEGIKTLQRAAKNAAKAGTVGKYAEAMAKNVEALL

SEQ ID NO: 53 - protein, 404 aa, synthetic construct
ADAASQVLLGSGLTILSQPLMYVKVLIQVGYEPLPPTIGRNIFGRQVCQLPGLFSYAQHIASIDGRRGLFTGLTPRLCSGVLGTVVHGKVLQHYQESDKGEELGPGNVQKEVSSSFDHVIKETTREMIARSAATLITHPFHVITLRSMVQFIGRESKYCGLCDSIITIYREEGILGFFAGLVPRLLGDILSLWLCNSLAYLVNTYALDSGVSTMNEMKSYSQAVTGFFASMLTYPFVLVSNLMAVNNCGLAGGCPPYSPIYTSWIDCWCMLQKEGNMSRGNSLFFRKVPFGKTYCCDLKMLIGGGGSAFEAAAARLEAEARALLARARARGPGRLYLVLQRELPSGTLETLASGDLERESLEETLARFRAALAAAPAGTYRAAVISLEPRLEVLESPPVEVEAP

SEQ ID NO: 54 - protein, 423 aa, synthetic construct
MKKIWLALAGLVLAFSASAADAASQVLLGSGLTILSQPLMYVKVLIQVGYEPLPPTIGRNIFGRQVCQLPGLFSYAQHIASIDGRRGLFTGLTPRLCSGVLGTVVHGKVLQHYQESDKGEELGPGNVQKEVSSSFDHVIKETTREMIARSAATLITHPFHVITLRSMVQFIGRESKYCGLCDSIITIYREEGILGFFAGLVPRLLGDILSLWLCNSLAYLVNTYALDSGVSTMNEMKSYSQAVTGFFASMLTYPFVLVSNLMAVNNCGLAGGCPPYSPIYTSWIDCWCMLQKEGNMSRGNSLFFRKVPFGKTYCCDLKMLIGGGGSAFEAAAARLEAEARALLARARARGPGRLYLVLQRELPSGTLETLASGDLERESLEETLARFRAALAAAPAGTYRAAVISLEPRLEVLESPPVEVEAP

SEQ ID NO: 55 - protein, 417 aa, synthetic construct
ADAASQVLLGSGLTILSQPLMYVKVLIQVGYEPLPPTIGRNIFGRQVCQLPGLFSYAQHIASIDGRRGLFTGLTPRLCSGVLGTVVHGKVLQHYQESDKGEELGPGNVQKEVSSSFDHVIKETTREMIARSAATLITHPFHVITLRSMVQFIGRESKYCGLCDSIITIYREEGILGFFAGLVPRLLGDILSLWLCNSLAYLVNTYALDSGVSTMNEMKSYSQAVTGFFASMLTYPFVLVSNLMAVNNCGLAGGCPPYSPIYTSWIDCWCMLQKEGNMSRGNSLFFRKVPFGKTYCCDLKMLIGGGGSDLEEFFEECLKEAEEVRKEVLEGKARIQIKESVRKEDPEGGTYVCGVISLGPKGGKFHVVFHKEDPEGKEETLARFEFKVEGGKVELLELKSKDPELKKLAEKYAEKLLE

SEQ ID NO: 56 - protein, 436 aa, synthetic construct
MKKIWLALAGLVLAFSASAADAASQVLLGSGLTILSQPLMYVKVLIQVGYEPLPPTIGRNIFGRQVCQLPGLFSYAQHIASIDGRRGLFTGLTPRLCSGVLGTVVHGKVLQHYQESDKGEELGPGNVQKEVSSSFDHVIKETTREMIARSAATLITHPFHVITLRSMVQFIGRESKYCGLCDSIITIYREEGILGFFAGLVPRLLGDILSLWLCNSLAYLVNTYALDSGVSTMNEMKSYSQAVTGFFASMLTYPFVLVSNLMAVNNCGLAGGCPPYSPIYTSWIDCWCMLQKEGNMSRGNSLFFRKVPFGKTYCCDLKMLIGGGGSDLEEFFEECLKEAEEVRKEVLEGKARIQIKESVRKEDPEGGTYVCGVISLGPKGGKFHVVFHKEDPEGKEETLARFEFKVEGGKVELLELKSKDPELKKLAEKYAEKLLE

SEQ ID NO: 57 - protein, 159 aa, synthetic construct
DLEEFFEECLKEAEEVRKEVLEGKARIQIKESVRKEDPEGGTYVCGVISLGPKGGKFHVVFHKEDPEGKEETLARFEFKVEGGKVELLELKSKDPELKKLAEKYAEKLLEGGGGSQAHELFRYFRMPELVDFRQYVRTLPTNTLMGFGAFAALTTFWYA

SEQ ID NO: 58 - protein, 178 aa, synthetic construct
MKKIWLALAGLVLAFSASADLEEFFEECLKEAEEVRKEVLEGKARIQIKESVRKEDPEGGTYVCGVISLGPKGGKFHVVFHKEDPEGKEETLARFEFKVEGGKVELLELKSKDPELKKLAEKYAEKLLEGGGGSQAHELFRYFRMPELVDFRQYVRTLPTNTLMGFGAFAALTTFWYA

SEQ ID NO: 59 - protein, 159 aa, synthetic construct; identical to SEQ ID NO: 13

SEQ ID NO: 60 - protein, 178 aa, synthetic construct
MKKIWLALAGLVLAFSASAQAHELFRYFRMPELVDFRQYVRTLPTNTLMGFGAFAALTTFWYAGGGGSDLEEFFEECLKEAEEVRKEVLEGKARIQIKESVRKEDPEGGTYVCGVISLGPKGGKFHVVFHKEDPEGKEETLARFEFKVEGGKVELLELKSKDPELKKLAEKYAEKLLE

SEQ ID NO: 61 - protein, 184 aa, Homo sapiens
GLASPDSPHDPCKMFIGGLSWQTTQEGLREYFGQFGEVKECLVMRDPLTKRSRGFGFVTFMDQAGVDKVLAQSRHELDSKTIDPKVAFPRRAQPKMVTRTKKIFVGGLSVNTTVEDVKQYFEQFGKVDDAMLMFDKTTNRHRGFGFVTFESEDIVEKVCEIHFHEINNKMVECKKAQPKEVMSP

SEQ ID NO: 62 - protein, 352 aa, synthetic construct
MKKIWLALAGLVLAFSASAGTVTLTVTVTATKDGEIKVSVMVRSSNKDVTLSISVSGGTLLNSSTATGSSGVAIGGATVYLKAGQTTTVTATATNVSADATVTASVGGDATFSSSTSTAGGGGSQAHELFRYFRMPELVDFRQYVRTLPTNTLMGFGAFAALTTFWYAGLASPDSPHDPCKMFIGGLSWQTTQEGLREYFGQFGEVKECLVMRDPLTKRSRGFGFVTFMDQAGVDKVLAQSRHELDSKTIDPKVAFPRRAQPKMVTRTKKIFVGGLSVNTTVEDVKQYFEQFGKVDDAMLMFDKTTNRHRGFGFVTFESEDIVEKVCEIHFHEINNKMVECKKAQPKEVMSP

SEQ ID NO: 63 - protein, 357 aa, synthetic construct
MKKIWLALAGLVLAFSASAGTVTLTVTVTATKDGEIKVSVMVRSSNKDVTLSISVSGGTLLNSSTATGSSGVAIGGATVYLKAGQTTTVTATATNVSADATVTASVGGDATFSSSTSTAGGGGSGLASPDSPHDPCKMFIGGLSWQTTQEGLREYFGQFGEVKECLVMRDPLTKRSRGFGFVTFMDQAGVDKVLAQSRHELDSKTIDPKVAFPRRAQPKMVTRTKKIFVGGLSVNTTVEDVKQYFEQFGKVDDAMLMFDKTTNRHRGFGFVTFESEDIVEKVCEIHFHEINNKMVECKKAQPKEVMSPGGGGSQAHELFRYFRMPELVDFRQYVRTLPTNTLMGFGAFAALTTFWYA

SEQ ID NO: 64 - protein, 258 aa, synthetic construct
MKKIWLALAGLVLAFSASAHHHHHHGLASPDSPHDPCKMFIGGLSWQTTQEGLREYFGQFGEVKECLVMRDPLTKRSRGFGFVTFMDQAGVDKVLAQSRHELDSKTIDPKVAFPRRAQPKMVTRTKKIFVGGLSVNTTVEDVKQYFEQFGKVDDAMLMFDKTTNRHRGFGFVTFESEDIVEKVCEIHFHEINNKMVECKKAQPKEVMSPGGGGSQAHELFRYFRMPELVDFRQYVRTLPTNTLMGFGAFAALTTFWYA